(19) United States
(12) Patent Application Publication
(10) Pub. No.: US 2023/0332207 A1
(43) Pub. Date: Oct. 19, 2023

(54) EPOXIDES FOR MULTIMODAL DETECTION OF BIOMOLECULES

(71) Applicant: Massachusetts Institute of Technology, Cambridge, MA (US)

(72) Inventors: Yi Cui, Cambridge, MA (US); Gaojie Yang, Cambridge, MA (US); Edward S. Boyden, Cambridge, MA (US)

(73) Assignee: Massachusetts Institute of Technology, Cambridge, MA (US)

(21) Appl. No.: 18/193,886

(22) Filed: Mar. 31, 2023

Related U.S. Application Data
(60) Provisional application No. 63/326,346, filed on Apr. 1, 2022.

(51) Int. Cl. C12Q 1/6806; C12Q 1/6837; C12Q 1/6825

(52) U.S. Cl. CPC C12Q 1/6806; C12Q 1/6837; C12Q 1/6825

(57) ABSTRACT

The invention relates, in part, to methods of unified expansion microscopy (uniExM) permitting multimodal detection of biomolecules.

### RELATED APPLICATIONS

This application claims benefit under 35 U.S.C. § 119(e) of U.S. Provisional application Ser. No. 63/326,346 filed Apr. 1, 2022, the disclosure of which is incorporated by reference herein in its entirety.

### GOVERNMENT SUPPORT

This invention was made with government support under EB024261 awarded by the National institutes of Health. The government has certain rights in the invention.

### FIELD OF THE INVENTION

The invention relates, in part, to methods of using epoxides in multimodal detection of biomolecules.

### BACKGROUND OF THE INVENTION

The advent of expansion microscopy (ExM) offers a unique solution to achieve super-resolution imaging by physically enlarging a specimen of interest. [Chen, F. et al., Science (2015) Vol. 347, 543-548; Tillberg, P W et al., Nat Biotechnol (2016) Vol. 34(9), 987-992; and Chen. F, et al., Nat Methods (2016) Vol. 13(8), 679-684] The working mechanism of ExM is comprised of three major steps: molecular anchoring, sample homogenization and isotropic expansion. [Wassie, A. T., et al. Nat Methods (2019) Vol. 16, 33-41; Tillberg, P. W. et al. Annu Rev Cell Dev Biol (2019) Vol. 35, 683-701; and Asano, S. M. et al. Curr Protoc Cell Biol (2018) Vol. 80, e56] In brief, a molecular anchor is first introduced to covalently bond with the target biomolecules proteins or nucleic acids), and then polyacrylate monomers are infused to create a swellable hydrogel network intertwined with the anchored biomolecules through free radical mediated polymerization. The formed hydrogel composite is subjected to heat denaturation or enzymatic digestion to free up rigid inter/intra-molecular connections (e.g., fixative crosslinks and peptide bonds) such that can be isotropically expanded in space upon dialysis with excessive amount of water.

ExM only requires chemicals and reagents that can be found in a common research laboratory, which helps the technology rapidly gain popularity idler the first report. To date, ExM has been successfully demonstrated in a wide range of sample types and given rise to a number of technical variants tackling diverse experimental purposes [see for example, Chozinski, T. J. et al. Nat Methods (2016) Vol. 13, 485-488, Zhao, Y. et al. Nat Biotechnol (2017) Vol. 35, 757-764; Chang, J. B. et al. Nat Methods (2017) Vol. 14, 593-599; Gao, R. et al. Science (2019) Vol. 363(6424); Truckenbrodt, S., et al. Nat Protoc (2019) Vol. 14, 832-863; Cahoon, C. K. et al. Proc Natl Acad Sci USA (2017) Vol. 114 E6857-E6866, Suofu, Y. et al. Proc Natl Acad Sci USA (2017) Vol. 114, E7997-E8006; Freifeld L. et al. Proc Natl Acad Sci USA (2017) Vol. 114, E10799-E10808; Shurer, C. R. et al. Cell (2019) Vol. 177, 1757-1770; Thevathasan, J. V. et al. Nat Methods (2019) Vol. 16, 1045-1053, Lim, Y. et al., PLoS Biol (2019) Vol. 17, e3000268; Xu, H. et al. Proc Natl Acad Sci USA (2019) Vol. 116, 18423-18428; Kao, P. et al., Sci Rep (2019) Vol. 9(17159), 1-11; So, C. et al. Science (2019) Vol. 364(6447); Hafner, A. S. et al., Science (2019) Vol. 364(6441); Halpern A. R. et al., ACS Nano (2017) Vol. 11, 12677-12686; Gao, M. et al., ACS Nano (2018) Vol. 12, 4178-4185; Li, R. et al., Nanoscale (2018) Vol. 10, 17552-17556; Gambarolto, D. et al., Nat Methods (2019) Vol. 16, 71-74; Karagiannis, E. D. et al. bioRxiv (2019), 829903; Ciao, R. et al. Nat Nanotechnol (2021), Vol. 16, 698-707; and M'Saad, O. et al., Nat Commun (2020). Vol. 11(3850), 1-15].

For instance, ExM and relevant techniques have been applied to study neuronal connectivity [Tillberg, P. W, et al. Nat Biotechnol (2016), Vol. 34(9), 987-992; Ku, T, et al., Nat Biotechnol (2016), Vol. 34, 973-981; and Shen, F. Y. et al., Natl Commun (2020), Vol. 11, 4632, 1-12]; synaptic ultrastructures [Hafner, A. S. et al., Science (2019), Vol. 364(6441); Mosca, T. J. et al., Elife (2017), Vol. 6(e27347), 1-29; and Sarkar, D. et al., bioRxiv (2020)]; chromosomal segregation [Cahoon, C. K. et al, Proc Natl Acad Sci USA (2017). Vol. 114, E6857-E6866; So, C. et al. Science (2019), Vol. 364(6447); Decarreau, J. et al., Nat Cell Biol (2017), Vol. 19, 384-390; and Decarreau, J. et al., Nat Cell Biol (2017), Vol. 19(740)]; tunneling nanotubes [Kumar, A. et al., Sci Rep (2017), Vol. 7, 40360 1-14]; stress granule formation [Cirillo, L. et al., Curr Biol (2020), Vol. 30, 698-707]; RNA localization [Chen, F. et al. Nat Methods (2016). Vol. 13(8), 679-684, Koppers, M. et. al., Elife (2019), Vol. 8(e48718) 1-27; and Coté, et al., bioRxiv (2020)]; spatial transcriptome [Wang, G. et al., Sci Rep (2018), Vol. 8(4847) 1-13; and Alon, S. et al., Science (2021), Vol. 371(6528)]; mitochondrial biology [Suolu, Y. et al. Proc Acad Sci USA (2017), Vol. 114, E7997-E8006; Fecher, C., et al., Nat Neurosci (2019), Vol. 22(10) 1731-1742; and Kurtz, T. C. et al., Front Cell Dev Biol (2020), Vol. 8(617) 1-10]; liquid-liquid phase separation [Falahati, H. et al., Soft Matter (2019), Vol. 15, 1135-1154]; and disease pathology [Zhao, Y. et al. Nat Biotechnol (2017), Vol. 35, 757-764]. To date, the majority of ExM protocols are designed to target only one class of biomolecules at a time, such as nucleic acids or proteins, while simultaneous detection of more than one molecules could substantially broaden ExM application. Development of better anchor molecules and methods will greatly extend the applicability of ExM techniques.

### REFERENCE TO AN ELECTRONIC SEQUENCE LISTING

The contents of the electronic sequence listing (sequencelisting.txt; Size: 1,816 bytes; and Date of Creation: Mar. 28, 2023) is herein incorporated by reference in its entirety.

### SUMMARY OF ELEMENTS OF INVENTION

According to an aspect of the invention, a method for preparing a biospecimen for multimodal detection of a biomolecule, the method including: (a) incubating a biological sample including the biomolecule with a multifunctional anchoring agent that covalently bonds the biomolecule, wherein the multifunctional anchoring agent includes an epoxide compound; (b) embedding the biological sample with anchored biomolecule in a polymer material; (c) homogenizing the embedded biological sample; and (d) physically expanding the homogenized embedded sample. in some embodiments, the biological sample is a clinical sample obtained from a subject, and optionally the clinical sample is a fixed clinical sample. In certain embodiments, the biological sample is a fixed biological sample. In some embodiments a means for fixing the biological sample includes an ethanol or a formaldehyde fixation method. In some embodiments, the biomolecule is a protein molecule, a nucleic acid molecule, a lipid molecule, a glycoprotein molecule, or a carbohydrate molecule. In certain embodiments, the biomolecule includes one or more of a protein molecule, a nucleic acid molecule, a lipid molecule, a glycoprotein molecule, and a carbohydrate molecule. In some embodiments, the method also includes performing the method on a plurality of the biomolecules. In some embodiments, the method also includes performing the method on a plurality of different biomolecules. In some embodiments, the method also includes polymerizing the polymer material. In certain embodiments, the method also includes performing the method on a plurality of different biomolecules. In certain embodiments, the epoxide compound is and/or includes an epoxide monomer. In some embodiments, the epoxide compound includes glycidyl methacrylate (GMA) and/or one or more other analogous acrylate epoxides. In some embodiments, the epoxide compound includes an epoxide group and an acrylate group. In certain embodiments, the epoxide compound includes an acrylate epoxide or epoxy acrylate. In some embodiments, the epoxide compound includes a precursor molecule that is processed, combined, or conjugated to include epoxide and acrylate groups. In some embodiments, a means for embedding the biomolecule in the polymer material includes incubating the biomolecule in a polymer monomer and polymerizing the monomer. In some embodiments, the polymer material includes as swellable polymer material. In certain embodiments, the swellable polymer material includes an acrylamide-co-acrylate copolymer. In certain embodiments, the polymer material includes a non-swellable polymer material capable of conversion to a swellable polymer material, and the method further includes converting the polymerized non-swellable polymer material into a swellable polymer material. In some embodiments, the method also includes converting the non-swellable polymer material into a swellable polymer material prior to the physically expanding step. In some embodiments, the non-swellable polymer material includes a non-swellable hydrogel. In certain embodiments, the non-swellable hydrogel includes one or more of an acrylamide and polyacrylate. In some embodiments, if the polymerized monomer is a swellable polymer, a means for the physical expansion of the biomolecule includes contacting the homogenized embedded biomolecule with a solvent or liquid that swells the swellable polymer. In certain embodiments, the liquid includes water. In some embodiments, as means of homogenizing the embedded biomolecule includes a heat denaturation method. In sonic embodiments, a means of homogenizing the embedded biomolecule includes contacting the polymer material in which the biomolecule is embedded with one or more of (i) a strong detergent or surfactant (e.g., sodium dodecyl sulfate) and (ii) one or more enzymes. In certain embodiments, the contacting enzyme is proteinase K (proK). In some embodiments, the contacting enzyme is an endoproteinase. In certain embodiments, the endoproteinase is LysC or Trypsin. In some embodiments, a means of physically expanding the biomolecule includes expanding the polymer material in which the biomolecule is embedded, wherein the expansion of the polymer material expands the homogenized biomolecule isotropically in at least a linear manner within the polymer material. In some embodiments, the polymer material includes a hydrogel and a means of expanding the hydrogel includes contacting the hydrogel with an aqueous solution, optionally water. In certain embodiments, the method also includes a passivating method. In some embodiments, the method also includes contacting the biomolecule with an antibody, an oligo probe or an affinity label capable of selectively binding the biomolecule at one or both of before or after the physical expansion step. In some embodiments, the method also includes attaching the biomolecule to a solid support prior to the embedding step. In certain embodiments, the solid support includes one or more of: polystyrene, polymethylmethacrylate (PMMA), polylysine, polyhistidine, glass, silica, metal, and plastic. In certain embodiments, the embedding step includes embedding the biomolecule attached to the solid support. In some embodiments, the method also includes cleaving the embedded biomolecule from the solid support. In some embodiments, a means for the cleaving includes contacting the embedded biomolecule with a reducing reagent, an enzyme, or photonic excitation. In certain embodiments, the reducing reagent is one or more of sodium cyanborohydride, dithiothreitol, β-mercaptoethanol, tris(2-carboxyethyl)phosphine and analogous chemicals that enables breakage of disulfide bonds. In some embodiments, the enzyme is capable of catalyzing breaking of covalent bonds. In some embodiments, the covalent bond is a peptide bond or a disulfide bond. In certain embodiments, the photonic excitation includes illumination with an energy or wavelength capable of cleaving a photosensitive linker. In certain embodiments, the method also includes detecting one or more of a spatial position, a structure, a component of, and an identity of the expanded biomolecule(s). In some embodiments, the physically expanded biomolecule is re-embedded in the same or a different polymer prior to the detecting. In certain embodiments, the re-embedding is in a non-swellable polymer. In some embodiments, the physically expanded biomolecule is not re-embedded in a polymer prior to the detecting. In some embodiments, a means for the detecting includes a method capable of capturing spatial data. In certain embodiments, a means for the detecting includes transferring the homogenized biomolecule from the polymer to a spatially indexed array, wherein the spatially indexed array optionally includes a microarray or a bead array. In some embodiments, a means for the detecting includes sectioning the expanded biomolecule, identifying the relative positions of the sections, recovering homogenized biomolecule material from the sections, detecting the homogenized biomolecule material, associating the detected homogenized biomolecule material with the identified relative positions of the homogenized biomolecule material versus the non-homogenized biomoleoule, and determining spatial positions of the associated detected homogenized biomolecule material. In some embodiments, the sectioning includes sectioning as an indexed grid. In some embodiments, the method also includes imaging the biomolecule(s) after the physical expansion. In certain embodiments, a means for the imaging includes optical microscopy. In some embodiments, the microscopy is light microscopy, fluorescence microscopy or electron microscopy. In some embodiments, the method also includes producing a high-resolution image of the biomolecule(s) after the physical expansion. In certain embodiments, a means of producing the high-resolution image includes imaging with an optical microscope. In certain embodiments, the method also includes detectably labeling the biomolecule. In some embodiments, a means for the detectably labeling includes directly or indirectly attaching one or more detectable labels to the biomolecule. In some embodiments, the detectably labeling includes affinity labeling, wherein optionally the affinity label includes one or more of biotin, digoxigenin, and a hapten. In certain embodiments, a means for the detectable labeling includes contacting the biomolecule with one or more enzymes, under suitable conditions for activity of the one or more enzymes to result in detectable labeling of biomolecule. In certain embodiments, the detectable label includes a fluorescent label, a luminescent label, a radiolabel, an enzymatic label, a contrast agent, a heavy metal, or a heavy element. In some embodiments, one or more of a detected spatial position, presence, absence, components of, and level of the biomolecule is associated with a disease or condition. In some embodiments, the method also includes classifying one or more of the detected spatial position, structure, and component of the expanded biomolecule(s) into one or more contiguous biomolecule molecule. In certain embodiments, a means of the classifying includes identifying the spatial positions of the detected homogenized biomolecule material in one or more dimensions and determining a relative ordering of the detected homogenized biomolecule material within a single contiguous biomolecule, wherein the relative ordering aids in classifying the detected homogenous biomolecule material into one or more contiguous biomolecules and identifying a structure of the ordered contiguous biomolecule. In some embodiments, the method also includes identifying one or more of a spatial position and a structural variation in the one or more classified contiguous biomolecules compared to a control structure. In some embodiments, one or more of the spatial position and the structural variation identified as present in the one or more biomolecules is associated with a disease or condition. In certain embodiments, the biomolecule is obtained from a cell. In some embodiments, the cell is obtained from a subject. In certain embodiments, the cell is a plant cell. In some embodiments, the subject is a mammal, optionally is a human. In some embodiments, the cell is a cultured cell. In certain embodiments, the cell is a fixed cell.

### BRIEF DESCRIPTION OF THE DRAWINGS.

FIG. 1A-B shows preservation of proteins, in tissues under high heat treatments by combinatorial application epoxides. Retention of Thy1-YFP fluorescence signals in mouse brain tissues by LabelX plus AcX, GMA plus TMPTE after: (FIG. 1A) detergent SDS-based denaturation: 95° C. for 1 h, followed by 37° C. overnight; (FIG. 1B) ProK-based digestion: 60° C. for 2 h, followed by 37° C. overnight. Scale bars (in pre-expansion units): 1000 μm (whole brain); 500 μm (half brain).

FIG. 2 shows results demonstrating GMA enables retention of proteins and RNAs in expanded cells and tissues. Antibody staining and HCR-FISH were performed post-expansion, respectively. Scale bars (in pre-expansion units): 20 μm.

FIG. 3A-B shows results demonstrating epoxide anchor enables unified Expansion Microscopy (uniExM). (FIG. 3A) provides a schematic diagram showing an embodiment of a standard ExM experiment, biomolecules are covalently bound to anchor molecules (epoxide as shown here) that can be incorporated into a swellable, polyacrylate network. After proper homogenization or digestion, the sample can be isotropically expanded upon dialysis with pure water. The versatility and potency of the anchor determine the molecular retention efficiency and multiplexing capacity of ExM. (FIG. 3B) Using the epoxide anchor GMA, simultaneous detection of nucleic acids and proteins was demonstrated across different sample types. In panel FIG. 3B(i), a 50 μm mouse brain tissue expressing Thy1-YFP was cut in half, one anchored with LabelX/AcX and the other with GMA. HCR-FISH targeting ACTB was performed and the FISH signals were detected together with the retained YFP signals after expansion. Scale bars (in pre-expansion units): 1000 μm (whole brain); 250 μm (hippocampus view). In FIG. 3B panel (ii), YFP and HCR-FISH spots intensities are compared between LabelX/AcX and GMA anchored mouse brain tissues (n=50 images, data points are presented in violin plots with mean values calculated). In FIG. 3B panel (iii), a variety of protein and RNA targets were co-detected in different samples anchored with GMA. Scale bars (in pre-expansion units): 20 μm.

FIG. 4 provides photomicrogaphs indicating that expansion helps reveal the periodic distribution of βII-spectrin in axons. Antibody staining βII-spectrin showed no specific structures before expansion, while the periodic, punctate distribution of βII-spectrin were detected after expansion. In comparison, antibody staining β-tubulin showed continuous microtubule structures even after expansion. Scale bars (in pre-expansion units): 20 μm (upper panels); 2 μm (lower panels).

FIG. 5A-B shows representative nucleophile substrates in biological systems, include (FIG. 5A) nucleic acids and (FIG. 5B) amino acids, Where their potential reactions with GMA are illustrated. In anchoring nucleic acids, the predominant reaction between GMA and N7-guanine is shown in shaded region.

FIG. 6A-B Shows results demonstrating characterization of GMA-based uniExM for protein and RNA retention. (FIG. 6A) uniExM improves imaging resolution and achieves homogenous expansion. Confocal images of HeLa cells stained against β-tubulin are presented. Before and after expansion, several key criteria of expansion were evaluated. Scale bars (in pre-expansion units): 20 μm (left image), 2 μm (middle and right images). FIGS. 6A(i) and (ii) show the cross-section intensity profiles highlighted by dark and light lines in the pre-expansion and post-expansion images, respectively. In FIG. 6A, panel axes of the same cell were measured before and after expansion (n=30 cells). The expansion factor was calculated to be 4.2. In FIG. 6B, panel (iv), RMS length measurement error was quantified with pre-expansion SoRa images versus post-expansion confocal images of HeLa cells (red line, mean; shaded area, standard deviation; n=5 samples). (FIG. 6B) (i) GMA-based expansion helps de-crowd densely packed mRNAs and better resolve single transcripts of the highly expressed GAPDH gene in HeLa cells. Scale bars (in pre-expansion units): 20 μm (left image), 2 μm (middle and right images). FIG. 6B(ii) GMA preserves all the RNA information during the expansion process. HCR-FISH targeting specific genes in same cells was performed before and after expansion. Transcripts were first counted before GMA anchoring and then the FISH probes were stripped off. Afterwards, GMA-based uniExM was performed, followed by HCR-FISH targeting the same genes. FIG. 6B(iii) shows results with three representative genes (from low to moderate expression levels) that were chosen and their transcript counts are presented (each spot represents reads front one single cell). Scale bars (in pre-expansion units): 5 μm.

FIG. 7 provides photomicrographic images and graphs showing the size and morphological descriptive of HeLa cell nuclei were assessed before and after GMA-based expansion (n=200 cells). Scale bars (in pre-expansion units): 50 μm.

FIG. 8A-D provides graphs demonstrating embodiments of optimization for GMA-based RNA anchoring. (FIG. 8A) Different concentrations of GMA, (FIG. 8B) reaction pH and (FIG. 8C) temperatures were tested in ExFISH targeting three housekeeping genes in HeLa cells, to find the optimal RNA retention condition. (FIG. 8D). The tunable GMA anchoring was demonstrated by adjusting the reaction temperature and pH. (n=50-80 cells for each tested condition).

FIG. 9A-B provides images and graphs show results of head-to-head comparison between GMA and LabelX in RNA detection. (FIG. 9A) ExFISH targeting three housekeeping genes was performed in HeLa cells treated with 0.04% GMA or 0.02% LabelX. Scale bars (in pre-expansion units): 20 μm. FIG. 9B) Summary of transcript counts in each single cell, (n=70-100 cells, two-sample t-test was performed, and the increased counts enabled by GMA and p values were presented on plots).

FIG. 10A-D provides images and graphs demonstrating preservation of intracellular fine structures by GMA anchoring. (FIG. 10A) Immunofluorescence staining against βII-spectrin was performed in cultured mouse hippocampal neurons with the standard expansion protocol (expansion factor ˜4×). Scale bar (in pre-expansion units): 1 μm. (FIG. 10B) The intensity profile across an axon segment is plotted. (FIG. 10C)(i) Immunofluorescence staining against βII -spectrin was performed in cultured hippocampus neurons with a modified expansion protocol (expansion factor ˜7×). The intensity profile across a distal axon segment is plotted. For axon segments with more than 10 spectrin rings, autocorrelation analysis was performed to calculate the periodicity value in space. (FIG. 10D)(i) The average autocorrelation function of periodicity analysis using 4× expansion. FIG. 10C(ii) and FIG. 10D(ii) show the averaged autocorrelation function of periodicity analysis using a modified 7× expansion protocol, (solid lines, mean value; shaded areas, standard error meaasured from 5 samples).

FIG. 11 provides a schematic illustration of targeted and untargeted ExSeq.

FIG. 12A-E provides images, a graph and result summary image that demonstrate application of GMA in ExSeq. (FIG. 12A) Amplicons generated by uExSeq in HeLa cells were detected with SBS reagents (from Illumina MiSeq kit). Scale bars (in pre-expansion units): 20 μm. (FIG. 12B) Representative images of ExFISH and targeted ExSeq detecting GAPDH mRNAs in HeLa cells. Scale bars (in pre-expansion units): 20 μm. (FIG. 12C) tExSeq against ACTB mRNA using “TTT” barcode containing padlock probes was performed in Thy1-YFP mouse brain tissues. Using the universal amplicon detection probe, the location of transcripts was highlighted. Next, in three rounds of sequencing reactions, signals from the same amplicons were detected in high fidelity. Scale bars (in pre-expansion units): 20 μm (for upper panel), 5 μm (for lower panel). (FIG. 12D) is a bar graph summarizing all the detected transcripts in the breast cancer PDX tissue. The numbered horizontal lines across the top identify transcript function: 1 Proliferation, 2 DNA repair, 3 Epithelial-mesenchymal transition (EMT), 4 Immune-signaling, 5 Metabolism, 6 Migration, 7 Transcription, and 8 Others. The transcripts for each of the vertical bars are: from left to right: KRAS, SOX4, BCL2, CDK12, AKT1, NF1, ARHGDIB, PIK3CA, AKT3, SEPT14, IGF2R, AKT2, EGER, BMP7, BRCA1, PARP1, BRCA2, PARP3, RAD21, ATM, FANCL, ATR, POLE, FANCD, POLQ, RAD51D, RAD50, VIM, SNA12, TWIST, NOTCH2, NCAD, CD44, EPCAM, SNAI1, HLA-C, B2M, CDKN2A, LGALS1, HLA-A, TXNL1, FBXO32, NFE2L2, SQLE, NDUFSS, CP, CRABP1, IDH2, SLC25A6, CTSV, HIF1A, COX5A, ALDH9A1, OAZ2, ANXA1, ARC, ATP5F1A, HACD3, S100A11, CTNNB1, CCDC88A, SPARC, TMSB15A, RNPS1, RBP1, CAMTA1, ENY2, ZNF24, ELOB, WDR61, SNRNP25, FOXL2, DDX24, SNRPA1, CDCA7, RPL17, RPS2, XIST, SEC11A, H1F0, UBE21, HSPE1, COL5A1, MORF4L1, MESD, IER31P1, and PSMA4. (FIG. 12E) Principal component analysis of the cancer related genes in this PDX sample identified two distinct tumor cell groups. 1-30 are: CP, CDK12, HIF1A, UBE2I, COX5A, IDH2, TMSB15A, SNRNP25, FANCD, SPARC, HLA-C, SEPT4, TWIST, RAD51D, CRABP1, COLSA1, H1F0, ARHGDIB, CD44, ANXA1, SOX4, ATR, MORF4L1, TXNL1, VIM, B2M, RPS2, NOTCH2, EPCAM, and XIST.

FIG. 13A-E provides schematic diagrams and images demonstrating use of epoxide anchor in expansion sequencing (ExSeq). (FIG. 13A) Schematic of targeted ExSeq. (FIG. 13B(i) Evaluation for in situ amplification of GMA-anchored mRNAs. The number of amplicons (targeting GAPDH mRNAs) per cell generated by ExSeq was compared with that by ExFISH in HeLa cells. (n 70 cells) FIG. 13B(ii) A raw fluorescence image showing high-SNR ExSeq signals from all four base channels. Scale bars (in pre-expansion units): 20 μm. (FIG. 13C) Application of ExSeq in breast cancer PDX tissues. FIG. 13C(i) Epoxide anchor largely reduces the cost of ExSeq experiments and so facilitate cost-effective spatial transcriptomics mapping. FIG. 13C(ii) 87 genes of various functional implication (presented as different shades) and significant cancer relevance were selected for measurement by targeted ExSeq. Scale bars (in pre expansion units): 100 μm (for entire tissue), 5 μm (for inset image). FIG. 13C(iii) Functional maps of gene groups were used to visualize distinct cell status and intra-tumor heterogeneity. Scale bars (in pre-expansion units): 100 μm. (FIG. 13D) Per principle component analysis (PCA), two major cell groups were identified. FIG. 13D(i) Using these PCA-related genes, unique distribution patterns of tumor cells were revealed. In this presented shaded graph, the combined counts of the top 15 genes in proportion to the total counts in each individual cell is assigned to the “Red” channel, while the combined counts of the bottom 15 genes in proportion to the total counts in each individual cell is assigned to the

“Green” channel. (color intensity scale: 0-30%) FIG. 13D(ii) In the selected region with mixed cell groups, 6 reprehensive genes were visualized to show the spatially varied expression. Scale bars (in pre-expansion units): 10 μm. (FIG. 13E) Uniform manifold approximation and projection (UMAP) representation of gene expression-based clustering reveals three main cell clusters, including two tumor cell clusters and one non-tumor cell cluster.

FIG. 14A-C provide images showing retention of lipids and carbohydrates by epoxide anchoring. (FIG. 14A)) Lipid staining reagents BODIPY, FM and R18 were tested in HeLa cells with both pre- and post-expansion staining, and the resulting images indicates successful anchoring of certain lipids content, if not all, to the polyacrylamide gel. Scale bars (in pre-expansion units): 20 μm. Orthogonal staining for lipids largely expands and enriches the biomolecular information ExM can obtain, such as mitochondria as shown in panel (FIG. 14B). Scale bars (in pre-expansion units): 20 μm (main images); 1 μm (insets). (FIG. 14C) WGA was used to stain glycoconjugates in HeLa cells post-expansion, which highlighted the GlcNAc-enriched structures such as plasma membrane (glycoproteins) and nuclear pores (nucleoporin). Scale bars (in pre-expansion units): 20 μm.

FIG. 15A-C provides photomicroscopic images demonstrating that epoxide anchoring enables multimodal imaging beyond proteins and RNAs. (FIG. 15A) Demonstration of simultaneous post-expansion staining for carbohydrates and lipids. (FIG. 15B) in addition to antibody staining for proteins and in situ hybridization for RNAs, anchoring of lipids and carbohydrates provides more biological information from the same cell, such as membranes, organelles (e.g., mitochondria and endoplasmic reticulum) and complex structures (e.g., nuclear pores). Scale bars (in pre-expansion units): 20 μm. (FIG. 15C) At the tissue level, multimodal detection by uniExM reveals the natural context where the biomolecules of interests reside. As shown here, the mouse brain tissue, WGA stains the blood vessels, HCR-FISH localizes mRNA transcripts, SMI-312 antibody stains neurofilaments, and DAPI stains cell nucleus. Scale bars (in pre-expansion units): 5 μm.

FIG. 16 l provides certain representative acrylate epoxides.

### DETAILED DESCRIPTION

The invention, w part, provides a cheap, fast, and multifunctional anchor system and technique that enables in situ quantification and characterization of biomolecules with super resolution. Embodiments of methods of the invention, termed unified Expansion Microscopy (uniExM), realize efficient and controllable preservation of a diversity of biomolecules utilizing acrylate epoxides as a standalone anchor molecule, The universal affordability and outstanding performance of uniExM pave the way for high-resolution spatial biology studies.

Methods of the invention provide an epoxide-based anchoring strategy for multiplexed molecular anchoring in ExM. The acrylate epoxide monomer utilized in embodiments of the invention features extremely low cost, ease of use, and high efficiency in anchoring diverse biomolecules. With this anchor molecule alone, it is now possible to achieve multivalent molecular preservation at the DNA, RNA, protein, lipid, and carbohydrate levels. As an additional advantage, the cost per unit for acrylate epoxides is million-fold cheaper than conventional anchor molecules used in previously established ExM protocols.

A multifunctional anchor for ExM that is chemically active, mechanistically predictable, functionally tunable, and universally accessible has now been discovered. Epoxide, which is also known as oxirane, is a cyclic ether that has a three-atom ring structure comprising oxygen attached to two adjacent carbon atoms. Epoxide is one of the most fundamental materials in daily life (e.g., epoxy adhesive and resin, surface paint and coating, and pharmaceutical ingredient). In certain embodiments of methods of the invention, acrydite epoxide is used as anchor. In some embodiments, glycidyl methacrylate (GMA) is used as acrylate epoxide anchor. In certain embodiments of methods of the invention, epoxides other than GMA may be used as an epoxide anchor.

The invention, in part, provides methods for obtaining a structure and identity of biomolecules. As used herein the term, “biomolecule” may be used in reference to a protein molecule, a lipid molecule, a glycoprotein molecule, a polynucleotide molecule, or a carbohydrate molecule. In some embodiments, a method of the invention is performed on a single type of biomolecule and in certain embodiments a method of the invention is performed on a plurality of a single type of biomolecule. For example, though not intended to be limiting, a single type of protein may be assessed using an embodiment of a method of the invention, and the method may be used on a plurality of the single type of protein molecule. In some embodiments, a method of the invention is performed on two or more different biomolecules. For example, though not intended to be limiting, a biological sample may include a plurality of different protein molecules and each may be assessed using a method of the invention. As another non limiting example, a biological sample may include one or more polynucleotide molecules and one or more protein molecules, each of which may he assessed using a method of the invention. As used herein, the term plurality means more than one, which may be 2, 3, 4, 5, 6, 7, 8, 9, 10, 11, 12, 13, 14, 15, or more. The terms “biospecimen” and “biological sample” are used interchangeably herein. The term “clinical sample” is used herein to mean a sample obtained from a subject.

Methods of the invention comprise modifying a biomolecule (or a plurality of biomolecules) with, an anchor molecule using an epoxide-based anchoring method as described herein, and then performing an ExM procedure on the anchored biomolecule. In sonic embodiments, the anchored biomolecule is embedded in a material. In some embodiments, the material comprises a swellable polymer material, and in certain embodiments of the invention, the material comprises a non-swellable polymer material that is capable of conversion to a swellable polymer material. Following embedding, the biomolecule undergoes controlled homogenization and the resulting homogenized. biomolecule material is physically expanded. In certain embodiments of methods in which the immobilized biomolecule material are embedded in a non-swellable material, the method may also include converting the non-swellable polymer material into a swellable polymer material prior to the physically expanding of the biomolecule material. Swelling of the material results in a physical expansion of the homogenized biomolecule material. Certain methods of the invention also include detecting the homogenized biomolecule materials using methods such as but not limited to hybridization and by enzymatic techniques, and the results of the detection provides one or both of structural and component information about the original biomolecule.

Methods of the invention, in part, include preparing biomolecules for enzymatic and microscopic analysis below the diffraction limit of light; to do this, embodiments of methods of the invention utilize a physical expansion of biomolecules in a polymer; a non-limiting example of which is a hydrogel. Unlike prior ExM methods, embodiments of the invention disclosed herein include use of an epoxide as a multifunctional anchor. In certain embodiments of methods of the invention, acrydite epoxide is used as anchor. In some embodiments, glycidyl methacrylate (GMA) is used as acrylate epoxide anchor. In certain embodiments of methods of the invention, epoxides other than GMA may be used as an epoxide anchor. It has now been determined that by using an epoxide as a multifunctional anchor permits recovery of spatial, structural, and identity information of multiple types of individual biomolecules in a biological sample.

In certain embodiments, methods include embedding one or a plurality of a biomolecule in a polymer, for example but not limited to an acrylamide polymer, followed by digestion, such as but not limited to proteolytic digestion, and swelling of the polymer comprising the embedded biomolecule(s), In certain embodiments, methods of the invention may be used for to assess a biomolecule of interest. For example, though not intended to be limiting, an embodiment of a method of the invention can be used to assess a protein in a biological sample, to detect and assess genomic DNA in a sample, to detect and assess a carbohydrate molecule(s) in a biological sample, etc. Methods of the invention can be used to detect and identify one or more alternations in proteins, lipids, glycoproteins, polynucleotides, etc. In a non-limiting example, an embodiment of a method of the invention may be used to identify and assess a polynucleotide (DNA or RNA) sequence such as, but not limited to: a genomic DNA sequence from a subject; a wild-type (control) genomic DNA sequence; a wild-type RNA sequence; a genetically modified RNA sequence; a genetically engineered genomic DNA sequence, a genomic DNA sequence or RNA sequence known to be or suspected of being associated with a disease or condition. Methods of the invention can be used to identify biomolecule components (e.g., amino acid sequences, nucleic acid sequences, etc.) and structures as well as differences in one or more biomolecules obtained from different sources. As a non-limiting example, methods of the invention may be used to compare structure and/or sequence/components of a normal (e.g., control) biomolecule to structure and/or sequence/components of a biomolecule obtained from a subject who has, or is suspected of having a disease or condition. Differences between the determined biomolecule and the control biomolecule may assist in identifying a biomolecule variation or abnormality associated with the subject's disease or condition. Methods of the invention are able to provide structure and component information beyond that obtainable from assessment of spatial localization of biomolecules when examined in unexpanded conformations.

### Polynucleotides/Proteins/Lipids/Carbohydrates/Glycoproteins

The term “nucleotide” as used herein includes a phosphoric ester of nucleoside—the basic structural unit of nucleic acids (DNA or RNA). The terms “polynucleotide”” and “nucleic acid” refer to a polymer comprising multiple nucleotide monomers and may be used interchangeably herein. A polynucleotide may be either single stranded, or double stranded with each strand having a 5′ end and a 3′ end. A nucleotide in a polynucleotide may be a natural nucleotide (deoxyribonucleotides A, T, C, or G for DNA, and ribonucleotides A, U, C, G for RNA)

The term “protein” and “polypeptide” refer to nitrogenous organic compounds comprising chains of amino acids and the terms may be used interchangeably herein.

The term “lipid” as used herein refers to organic compounds comprising fatty acids or their derivatives.

The term “carbohydrate” refers to a molecule that includes carbon (C), hydrogen (H) and oxygen (O) atoms. Another term for carbohydrate is saccharide, which is a group that includes sugars, starch, and cellulose. Monosaccharides and disaccharides are relatively low molecular weight carbohydrates. Larger saccharides include polysaccharides and oligosaccharides:

The term “glycoprotein” as used herein refers to any of a class of proteins that have carbohydrate groups attached to a polypeptide chain. A glycoprotein comprises oligosaccharide chains (glycans) that are covalently attached to amino acid side-chains.

In some embodiments, a biomolecule assessed using a method of the invention is a “modified biomolecule”, which, as used herein, refers to a biomolecule that comprises one or more non-natural or derivatized components. As used herein the term “component” means a portion of the biomolecule. As non-limiting examples; the term “component” used in reference to (1) a protein biomolecule may be an amino acid, (2) a polynucleotide biomolecule may be a nucleic acid; (3) a lipid biomolecule may be a fatty acid; (4) a carbohydrate molecule may be a saccharide or polysaccharide; and (5) a glycoprotein molecule may be a carbohydrate molecule, an amino acid, or a protein molecule. In some embodiments, a component of a biomolecule is chemically or biochemically modified. In some embodiments of the invention, one or more modified components are incorporated into a biomolecule. Modified biomolecules may confer desirable properties absent or lacking in the natural biomolecule and biomolecules comprising one or more modified components may be used in the compositions and methods of the invention. As used herein, a “modified biomolecule” refers to a biomolecule comprising at least one modified component. In some embodiments, a modified biomolecule may comprise one, two, three, four, five, or more modified components.

A polynucleotide may be DNA (including but not limited to cDNA or genomic DNA), RNA, or hybrid polymers (e.g., DNA/RNA). The terms “polynucleotide” and “nucleic acid” do not refer to any particular length of polymer. Polynucleotides used in embodiments of methods of the invention may be at least 1, 2, 3, 4, 5,10, 20, 30, 40, 50, 60, 70, 80, 90, 100, 200, 500, 1000, 2000, or 5000 kb or more in length. The term “protein” does not refer to any particular length of the molecule. A protein used in embodiments of methods of the invention may be at least 1, 5, 10, 20, 30, 40, 50, 60, 70, 80, 90, 100, 200, 500, or more amino acids in length

The term “sequence,” used herein in reference to a polynucleotide or protein, refers to a contiguous series of nucleotides or amino acids, respectively. The term “structure” as used herein in reference to a polynucleotide refers to overall sequence organization of the polynucleotide, including “structural variations” such as insertions, deletions, repeats, and rearrangements. A polynucleotide or other biomolecule may be chemically or biochemically synthesized, or may be isolated from a subject, cell, tissue, or other source or sample that comprises, or is believed to comprise, the biomolecule. The term “structure” as used herein in reference to a protein, lipid, or glycoprotein refers to overall s component organization of the biomolecule. A biomolecule used in a method of the invention may be chemically or biochemically synthesized, or may be isolated from a subject, cell, tissue, or other source or sample that comprises, or is believed to comprise, one or more biomolecules of interest.

### Anchoring

Methods of the invention include anchoring a biomolecule to a multifunctional anchoring agent. In some embodiments of the invention, a means for the anchoring comprises incubating the biological sample comprising the biomolecule with a multifunctional anchoring agent that covalently bonds to the biomolecule. It has now been determined that using an epoxide as a multifunctional anchor permits recovery of spatial, structural, and identity information of multiple types of individual biomolecules in a biological sample. In some embodiments, the multifunctional anchoring agent is an epoxide, which may also be referred to herein as an “epoxide compound.” In certain embodiments, the epoxide compound is an epoxide monomer. In certain embodiments of methods of the invention, acrydite epoxide is used as the multifunctional anchoring agent. In some embodiments, an acrylate epoxide multifunctional anchoring agent comprises glycidyl methacrylate (GMA). In some embodiments, a multifunctional anchoring agent is GMA. In certain embodiments of methods of the invention, epoxides other than GMA may be used as an epoxide anchor. Additional acrylate epoxides, also referred to herein as analogous acrylate epoxides, are known in the art, and may be used as multifunctional anchoring agents in certain embodiments of the invention. In some embodiments, an epoxide compound used in a method of the invention comprises a precursor molecule that is processed, combined, or conjugated to include epoxide and acrylate groups. Non-limiting examples of epoxy acrylate compounds that may be used in methods of the invention are shown in Table 1. Preparation and use of epoxide compounds that may be used in methods of the invention are described in publications see for example, Bednarczyk, P. et al. 2021, 13, 1718. doi.org/10.3390/polym13111718, which is incorporated by reference herein in its entirety.

TABLE 1
Certain representative acrylate epoxides

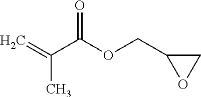

Glycidyl methacrylate

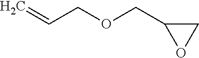

Allyl glycidyl ether

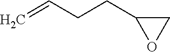

1,2-Epoxy-5-hexene

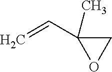

2-Methyl-2-vinyloxirane

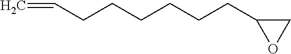

1,2-Epoxy-9-decene

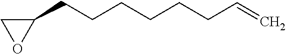

(R)-(+)-1,2-Epoxy-9-decene

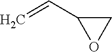

3,4-Epoxy-1-butene

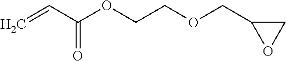

4-Hydroxybutyl acrylate glycidyl ether

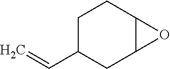

4-Vinyl-1-cyclohexene 1,2-epoxide

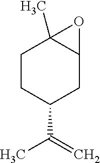

(+)-Limonene 1,2-epoxide

### Free and/or Immobilization

Methods of the invention may be used on biomolecules free in solution or immobilized on a solid support. As used herein, a “solid support” means one or more of a polystyrene, a polymethylmethacrylate (PMMA), a polylysine, a polyhistidine, a glass, a silica, a metal, a plastic, a vinyl silane, an aminosilane, or a PDMS surface (see, for example, U.S. Pat. No. 5,840,862, which is incorporated by reference herein in its entirety). Immobilization methods may include, but are not limited to: nonspecific adhesion due to heat, or a fixation method such as ethanol or formaldehyde fixation.

### Embedding

Embodiments of methods at the invention may include embedding the biomolecule in a polymer material. In some instances, a means for embedding the biomolecule in the polymer material comprises incubating the biomolecule in a polymer monomer and polymerizing the monomer. In some embodiments, the polymer material is a swellable polymer material, A non-limiting example of a swellable polymer material comprises an acrylamide-co-acrylate copolymer. As used herein, the term. “swellable polymer material” generally refers to a material that expands when contacted with a liquid, such as water or other solvent [Wassie A:, et al., Nat. Methods 16, 33-41 (2019) and U.S. Pat. No. 10,059,990 in relation to swellable and non-swellable materials, each publication is incorporated by reference herein in its entirety.]

The swellable material may uniformly expand in three dimensions. Additionally or alternatively, the material is transparent such that, upon expansion, light can pass through the sample. In some embodiments, the swellable polymer material is a swellable polymer or hydrogel. In one embodiment, the swellable polymer is formed in situ from precursors thereof: for example, one or more polymerizable materials, monomers or oligomers may be used, such as monomers selected from the group consisting of water-soluble groups containing a polymerizable ethylenically unsaturated group. Monomers or oligomers may comprise one or more substituted or unsubstituted methacrylates, acrylates, acrylamides, methacrylamides, vinylalcohols, vinylamines, allylamines, allylalcohols, including divinylic crosslinkers thereof (e.g., N,N-alkylene bisacrylamides). Precursors may also comprise polymerization initiators and crosslinkers.

In some embodiments, a swellable polymer is an acrylamide-co-acrylate copolymer, polyacrylate, or polyacrylamide, or co-polymers or cross-linked co-polymers thereof. Alternatively or additionally, the swellable polymer may be formed in situ by chemically cross-linking water-soluble oligomers or polymers. Thus, the invention envisions adding precursors, such as water-soluble precursors, of the swellable polymer to the sample and rendering the precursors swellable in situ.

Certain embodiments of the invention include embedding a biomolecule in a non-swellable polymer material capable of conversion to a swellable polymer material. In this instance, the method may also include polymerizing the non-swellable polymer and then converting the polymerized non-swellable polymer material into a swellable polymer material. As used herein, the term “non-swellable polymer material” comprises a polymer material capable of conversion to a swellable polymer material, including a non-swellable hydrogel comprising one or more of an acrylamide and polyacrylate [Ueda H., et al., Nat. Rev. Neurosci, 21, 61-79 (2020)]. In some embodiments of the invention, the polymer is not a polyacrylade polymer. In some embodiments of methods of the invention, the non-swellable polymer material is converted into a swellable polymer material before the physical expanding step of the method. A non-swellable polymer can comprise various materials. As a non-limiting example, a non-swellable, polymer material may include a non-swellable hydrogel. As another non-limiting example, a non-swellable hydrogel may include one or more of an acrylamide and polyacrylate. A non-swellable polymer used in an embodiment of a method of the invention may be a polymer that can be chemically converted into a swellable polymer. For example, such a non-swellable polymer may be acrylamide; acrylamide can later be converted into an acrylamide-co-acrylate copolymer after treatment with a strong base such as sodium hydroxide, which can then swell after dialysis with water. Other polymers such as polyacrylate may also be used in certain embodiments of the invention.

In some embodiments of methods of the invention, a non-swellable or swellable polymer may be cast in a thin overlay over a solid support, and may bind to the solid support when the support itself has reactive groups that can participate in free radical polymerization, or otherwise nonspecifically bind the gel, as is the case, for example, with a vinyl silane surface and aminosilane surface respectively.

### Homogenization

Embodiments of methods of the invention include a homogenization step. As used herein and in the expansion microscopy arts, the term “homogenization” refers to a process that frees up, also referred to as “releases” intra-sample connections before expansion. For example, a homogenization step may be used to release connections within the biomolecule. The release of connections loosens the biomolecule in place and renders the biomolecule capable of expanding in the expansion step of the method. A term for a biomolecule following a homogenization step in a method of the invention is “homogenized biomolecule material,” which indicates the biomolecule has been homogenized and is in a condition in which the biomolecule is capable of expansion. In some embodiments of methods of the invention, a means of homogenization of a biomolecule includes one or more of an enzyme-based digestion and a heat-based denaturation. In some embodiments of methods of the invention, a means of homogenizing an embedded biomolecule comprises contacting the polymer material in which the biomolecule is embedded with one or more of (i) a strong detergent or surfactant (e.g., sodium dodecyl sulfate); (ii) one or more enzymes; and (iii) denaturing heat. Non-limiting examples of enzymes that may be used in a homogenization step of a method of the invention are: proteinase K (proK) an endoproteinase, non-limiting examples of which are: LysC and trypsin.

### Surface Detachment

In embodiments in which a biomolecule is attached to a solid support, a surface detachment step may be included in an embodiment of a method of the invention. Once the biomolecule has been embedded and immobilized in a polymer overlay, methods of the invention may include steps of surface detachment, homogenization, and hydrogel conversion. As used herein, “cleaving ” or “surface detachment” mean that the polymer overlay is cleaved from the solid support such that the embedded biomolecule remains in the gel phase rather than adhering to the support. Means for cleaving the polymer overlay from the solid support include contacting the support and embedded biomolecule with one or more of: a reducing reagent, an enzyme, or photonic excitation. In some embodiments, the reducing reagent is sodium cyanoborohydride, dithiothreitol, β-mercaptoethanol, tris(2-carboxyethyl)phosphine, or other analogous chemicals capable of breaking disulfide bonds. In embodiments of the invention that includes use of an enzyme for cleaving/surface detachment, the enzyme is an enzyme capable of catalyzing breaking of covalent bonds. Non-limiting examples of covalent bonds that the enzyme is capable of breaking are a peptide bond or a disulfide bond. In embodiments of the invention that include use of photonic excitation for cleavage/surface detachment, the photonic excitation may include illumination of the support and polymer in which the biomolecule is embedded with an energy or wavelength capable of cleaving a photosensitive linker.

### Polymer and Biomolecule Physical Expansion

The polymer within which homogenized biomolecule materials are embedded is isotropically expanded, In some embodiments, a solvent or liquid is added to the polymer containing the homogenized biomolecule material and the solvent or liquid is absorbed by the swellable material and causes swelling. For example, if the mechanism of expansion is the polyelectrolyte effect, the polymer may be dialyzed against water or an aqueous solution to expand. In one embodiment, the addition of water allows the embedded sample to expand at least 3, 4, 5, or more times its original size in three dimensions. Thus, the sample may be increased 100-fold or more in volume.

In some embodiments of methods of the invention, a means of physically expanding the biomolecule includes expanding the polymer material in which the biomolecule is embedded, wherein the expansion of the polymer material expands the homogenized biomolecule isotropically in at least a linear manner within the polymer material. In certain embodiments, the polymer material comprises a hydrogel and a means of expanding the hydrogel includes contacting the hydrogel with an aqueous solution, optionally water.

Certain embodiments of the invention an expanded swellable polymer comprising homogenized biomolecule material may be re-embedded in a non-swellable or in a swellable polymer prior to detection of the biomolecule material. A re-embedded swellable polymer may be partially or completely degraded chemically, provided the biomolecule material in the polymer either remains anchored or is transferred to the non-swellable polymer. In some embodiments of the invention, non-charged polymer chemistries may be used to avoid charge passivation. In certain embodiments of the invention, the physically expanded polymer and biomolecule materials are not re-embedded in a polymer prior to being detected.

### Passivating

Certain embodiments of methods of the invention may also include a passivating step. As used herein the terms “passivating” or ““passivation” refer to a process for rendering a polymer material less reactive with components contained within the polymer material. In sonic embodiments of the invention passivation of a polymer comprising a biomolecule is used to reduce and/or prevent unwanted downstream enzymatic reactions. A non-limiting example of passivation of a polymer material is functionalizing the polymer material with one or more chemical reagents to neutralize charges within the polymer material. In some embodiments of the invention, a swellable polymer containing expanded biomolecule material is not passivated.

### Labelling

A biomolecule or biomolecule material embedded in a polymer may be “labelled” or “tagged” with a detectable label. As used herein, the term “detectable label” means a label or tag that is chemically bound to the biomolecule or to a component thereof, through covalent, hydrogen, or ionic bonding, and is detected using microscopy or one or more other means of detection. A detectable label may be selective for a specific target (e.g., a biomarker or class of molecule), as may be accomplished with an antibody or other target specific binder, or the detectable label may be an affinity label, including one or more of biotin, digoxigenin, and a hapten. In some embodiments, a detectable label comprises a visible component, as is typical of a dye or fluorescent molecule, a luminescent label, a radiolabel, an enzymatic label, a contrast agent, a heavy metal, or a heavy element such as bromine or iodine, or metals such as gold, osmium, rhenium, etc.; however any signaling means used by the label is also contemplated. A fluorescently labeled polynucleotide, protein, carbohydrate, glycoprotein, or lipid biomolecule, for example, is a polynucleotide, protein, carbohydrate, glycoprotein, or lipid biomolecule, respectively that is labeled through techniques such as, but not limited to, immunofluorescence, immunohistochemical or immunocytochemical staining to assist in microscopic analysis.

In some embodiments, the detectable label is a probe, antibody, and/or fluorescent dye, wherein the antibody and/or fluorescent dye further comprises a physical, biological, or chemical anchor or moiety that attaches or crosslinks the sample to the composition, polymer (e.g., hydrogel), or other swellable material. The detectable label may be attached to the nucleic acid adaptor, and in some embodiments, more than one label may be used. For example, each label may have a particular or distinguishable fluorescent property, e.g., distinguishable excitation and emission wavelengths. Further, each label may have a different target-specific binder that is selective for a specific and distinguishable target in, or component of the sample. In other embodiments, the detectable label is indirectly attached to the biomolecule by means of hybridizing one or more detectably labelled probes to the biomolecule material or component, such as fluorescently labelled DNA probes, a detectably labelled antibody, etc.

In other embodiments, enzymatic methods for detectable labeling are used, including contacting the biomolecule and/or biomolecule material with one or more enzymes, under suitable conditions for activity of the one or more enzymes to result in detectable labeling of biomolecule and/or biomolecule material, respectively.

### Detecting Structure and Sequence

Methods of the invention allow detection of spatial structures and components of expanded biomolecule material using microscopic and enzymatic detection methods. The signal from individual molecules may be spatially punctate due to the homogenization step. However, these puncta will be spatially proximal, allowing the overall length of the biomolecule to be inferred based on the dimension in which spatial proximity is highest. Thus, the structure of the biomolecule may be inferred over distances up to the entire length of the biomolecule.

As used herein, “detecting” means using one or both of an imaging method and a sequencing method to identify the spatial position and components of biomolecules. Imaging methods include but are not limited to light microscopy, epi-fluorescence microscopy, confocal microscopy, spinning disk microscopy, multi-photon microscopy, light-sheet microscopy, total internal reflection (TIRF) microscopy, Light-field microscopy, Imaging mass spectrometry, Imaging Raman spectroscopy, super-resolution microscopy, or transmission electron microscopy. Enzymatic detection methods include but are not limited to random primer extension, terminal transferase tailing, padlock probe rolling circle amplification [Larsson, C., et al, Nat. Methods 1(3): 227-232: (2004)], in situ PCR [Hodson, R., et al. Appl. Environ. Microbiol. 4074-4082 (1995)], horseradish peroxidase tyramide signal amplification [Schonhuber, W. et al. Appl. Environ. Microbiol. 3268-3273 (1997)], luciferase-catalyzed pyrophosphate chemiluminescence [Nyren, P., et al. Anal. Biochem. 208:171-175 (1993)], or other PCR-based or DNA sequencing methods [Stãhl, P. L., et al. Science 353.6294: 78-82 (2016); Rodrigues, S, G., et al., Science 363.6434: 1463-1467 (2019)].

As used herein, “spatial position” refers to the location of a biomolecule, biomolecule material, and/or biomolecule component relative to the location of another biomolecule, biomolecule material, and or biomolecule component, respectively. Certain embodiments of methods of the invention are useful to determine relative positions of one or more components or biomolecule materials generated from a single biomolecule. For example, a biomolecule component generated from a protein biomolecule might he an amino acid, a peptide and methods of the invention can be used to determine the relative positions of these components in the original protein biomolecule. Thus, embodiments of methods of the invention can be used to disarticulate a biomolecule into components in a controlled manner and then to identify the components and elative positions of the resulting components in the expanded conformation.

An additional aspect of the invention is that the biomolecule may be transferred from a solid phase support to a quasi-liquid-phase hydrogel, which is >99% liquid phase. Because many enzymatic reactions are inefficient on solid phase supports, it may be efficient to analyze the biomolecule after removal from a support. In specific circumstances, a judicious choice of surface and technique permits the possibility of enzymatic reaction, and other reactions useful to assess and identify components and spatial positions of components of biomolecules.

In some embodiments of methods of the invention, a means for detecting may comprise transferring the components of a biomolecule from the polymer to a spatially indexed array, wherein the spatially indexed array optionally comprises a microarray or a bead array in order to capture spatial data. In some embodiments methods of the invention, a means for the detecting comprises one or more of: sectioning the expanded gel, identifying the relative positions of the sections, recovering biomolecule components and/or material from the sections, detecting the biomolecule components and/or material, associating the detected components and/or material with their identified relative positions, and determining the spatial positions and identity of the associated detected components and/or materials [Kebschull, J. M., et al., Neuron 91.5: 975-987 (2016)]. In some embodiments, the expanded gel may be sectioned as an indexed grid. A non-limiting example of an embodiment of the invention utilizing an indexed grid includes sectioning the polymer into pieces using e.g., a knife, keeping track of (indexing) the relative positions of the sections. These sections are then processed independently (i.e., through DNA retrieval and conventional sequencing, amino acid detection, etc.), and the relative positions of components and/or materials of the biomolecule in one section relative to other sections can then be reconstructed.

### Analysis

Certain embodiments of methods of the invention may be used to analyze structure, spatial organization, and sequence of one or more biomolecules of interest that may be known or may be suspected of being associated with a disease or condition. Some embodiments of methods of the invention can be used to identify a biomolecule associated with a disease or condition. Non-limiting examples of diseases and conditions that can be assessed using embodiments of the invention are: disease and conditions such as but not limited to: foodborne illness, food poisoning associated conditions; bacterial infection; viral infections; parasitic infections; poisoning; contamination end/or poisoning with one or more toxins and heavy metals; sickle cell anemia; hemophilia; cystic fibrosis; Tay Sachs disease; Huntington's disease; fragile X syndrome; chromosomal disorders such as but not limited to: Down syndrome and Turner syndrome; polygenic disorders such as but not limited to Alzheimer's disease, heart disease, cancers, and diabetes, etc. Methods of the invention can also be used in forensic examination.

### Subjects and Cells

The term “subject” may refer to human or non-human animals, including mammals and non-mammals, vertebrates and invertebrates, and may also be any multicellular organism or single-celled organism such as a eukaryotic. (including plants and algae) or prokaryotic organism, archaeon, microorganisms (e.g., bacteria, archaea, fungi, protists, viruses), and aquatic plankton. A subject may be considered a normal subject or may be a subject known to have or suspected of having a disease or condition. In some embodiments, an organism is a genetically modified organism. In some embodiments, a subject is a plant. As used herein the term “genetically modified” is used interchangeably with the term “genetically engineered”.

Cells, tissues, or other sources or samples may include a single cell, a variety of cells, or organelles. It will be understood that a cell sample comprises a plurality of cells. As used herein, the term “plurality” means more than one. In some instances, a plurality of cells is at least 1, 10, 100, 1,000, 10,000, 100,000, 500,000, 1,000,000, 5,000,000, or more cells. A plurality of cells from which biomolecules are obtained for use in methods of the invention may be a population of cells. A plurality of cells may include cells that are of the same cell type. In some embodiments, a cell from which one or more biomolecules are obtained for use in methods of the invention is a healthy normal cell, which is not known to have a disease, disorder, or abnormal condition. In some embodiments, a plurality of cells from which biomolecules are isolated for use in methods of the invention includes cells having a known or suspected disease or condition or other abnormality, for example, a cell obtained from a subject diagnosed as having a disorder, disease, or condition, including, but not limited to a degenerative cell, a neurological disease-bearing cell, a cell model of a disease or condition, an injured cell, etc. In some embodiments, a cell is an abnormal cell obtained from cell culture, a cell line known to include a disorder, disease, or condition. Non-limiting examples of diseases or conditions include disorders, such as sickle cell anemia, hemophilia, cystic fibrosis, Tay Sachs disease, Huntington's disease, and fragile X syndrome; chromosomal disorders, such as Down syndrome and Turner syndrome, Alzheimer's disease, heart disease, diabetes; and cancers.

In some embodiments of the invention, a plurality of cells is a mixed population of cells, meaning all cells are not of the same cell type. Cells may be obtained from any organ or tissue of interest, including but not limited to skin, lung, cartilage brain, CNS, PNS, breast, blood, blood vessel (e.g., artery or vein), fat, pancreas, liver, muscle, gastrointestinal tract, heart, bladder, kidney, urethra, and prostate gland. In some embodiments, a cell from which one of more biomolecules are isolated for use in methods of the invention is a control cell. In various embodiments, cells from which one or more biomolecules are isolated for use in methods of the invention may be genetically modified or not genetically modified.

A cell from which one or more biomolecules are obtained for use in methods of the invention may be obtained from a biological sample obtained directly from a subject. Non-limiting examples of biological samples are samples of: blood, saliva, lymph, cerebrospinal fluid, vitreous humor, aqueous humor, mucous, tissue, surgical specimen, biopsy specimen, tissue explant, organ culture, biological fluid or any other tissue or cell preparation, or fraction or derivative thereof or isolated therefrom, etc. In some embodiments of the invention, one or more biomolecules may be obtained from primary cells, cell lines, freshly isolated cells or tissues, frozen cells or tissues, paraffin embedded cells or tissues, fixed cells or tissues, and/or laser dissected cells or tissues. In some embodiments, a sample from which one or more biomolecules are isolated for use in methods of the invention is a control sample. Biomolecules may be isolated from a subject, cell, or other source according to methods known in the art. A cell or subject from which a biomolecule is obtained for use in an embodiment of a method of the invention may be a genetically engineered cell or subject, respectively.

### EXAMPLES

### Example

### Methods and Materials

### Cell Culture and Mouse Brain Tissues

HeLa cells were routinely cultured in DMEM medium supplemented with 10% FBS and 1% antibiotics. Once the cells reached 70-80% confluency, they were fixed with either 4% paraformaldehyde (PFA) or 3% PFA/0.1 glutaraldehyde (GA, for better preservation of intracellular fine structures including microtubules and spectrins), followed by residual aldehyde quenching with 0.1% sodium borohydride and 100 glycine. For RNA detection ExFISH and ExSeq), samples were permeabilized with 70% ethanol at 4° C. overnight and stored up to 4 weeks.

Primary neurons were dissected from newborn Swiss Webster mice and about 1,000 hippocampal neurons were seeded onto a 12 mm #1.5 coverslip. The neurons were further cultured for 2 weeks and then fixed for subsequent uses.

For mouse brain tissues, seven-week old mice were terminally anesthetized with isoflurane and euthanized by decapitation, followed by transcardial perfusion with PBS and ice cold 4% PFA. Then the brain was dissected out and placed in 4% PFA for 12-16 hours. 50 μm slices were prepared on a vibratome (Leica VT1000s) and then stored at 4° C. in PBS or 70% ethanol until use.

### Patient-Derived Xenografts (PDX)

Patient tumor samples were acquired with informed consent, according to procedures approved by the Ethics Committees at the University of British Columbia. Breast cancer patients undergoing diagnostic biopsy or surgery were recruited and samples collected under protocols H06-00289 (BCCA-TTR-BREAST), H11 -01887 (Neoadjuvant Xenograft Study), H18-01113 (Large-scale genomic analysis of human tumors) or H20-00170 (Linking clonal genomes to tumor evolution and therapeutics). Tumor fragments were finely Chopped and mechanically disaggregated for one minute using a Stomacher 80 Biomaster (Seward Limited, Worthing UK) 1 mL cold DMEM/F-12 with Glucose, L-Glutamine and HEPES (Lonza 12-719F). 200 mL of medium containing cells/organoids from the suspension was used for transplantation per mouse. Tumors were transplanted in mice as previously described in accordance with SOP BCCRC 009 [Eirew, P. et al. Nature (2015), Vol. 518, 422-426]. Female NOD/SCID/IL2Rγ−/− (NSG) and NOD/Rag1−/−Il2Rγ31 /− (NRG) mice were bred and housed at the Animal Resource Centre at the British Columbia (BC) Cancer Research Centre. Disaggregated cells and organoids were resuspended in 150-200 μl of a 1:1 v/v mixture of cold DMEM/F12; Matrigel (BD Biosciences, San Jose, Calif., USA). 8-12-week-old mice were anesthetized with isoflurane and the suspension was transplanted under the skin on the left flank using a 1 mL syringe and 21-gauge needle. The animal care Female NOD/SCID/IL2Rγ−/− (NSG) and NOD/Rag1−/−Il2Rγ−/− (NRG) mice were bred and housed at the Animal Resource Centre at the British Columbia (BC) Cancer Research Centre. Disaggregated cells and organoids were resuspended in 150-200 μl of a 1:1 v/v mixture of cold DMEM/F12; Matrigel (BD Biosciences, San Jose, Calif., USA). 8-12-week-old mice were anesthetized with isoflurane and the suspension was transplanted under the skin on the left flank using a 1 mL syringe and 21-gauge needle. The animal care committee and animal welfare and ethical review committee, the University of British Columbia (UBC), approved all experimental procedures. Tables 2 and 3 provide information on antibodies and chemicals used in experiments described herein.

TABLE 2
Certain antibodies used in
studies described herein

| | Vendor | Catalog |
|---|---|---|
| Antibody name | information | number |
| Mouse anti- | DSHB | E7 |
| tubulin | | |
| Rabbit anti-GFP | ThermoFisher | A-6455 |
| | Scientific | |
| Mouse anti-βII- | BD | 612563 |
| spectrin | Biosciences | |
| Mouse anti- | BioLegend | 801801 |
| MAP2 | | |
| Mouse anti- | BioLegend | 837904 |
| SM [symbol] 312 | | |
| Goat anti-mouse | ThermoFisher | A-21430 |
| IgG Alexa 488 | Scientific | |
| Goat anti-rabbit | ThermoFisher | A-11017 |
| IgG Alexa 555 | Scientific | |
| Goat anti-mouse | ThermoFisher | A-11018 |
| IgG Alexa 546 | Scientific | |
| Goat anti-rabbit | ThermoFisher | A-21246 |
| IgG Alexa 647 | Scientific | |

[symbol]

indicates data missing or illegible when filed

TABLE 3
Certain chemicals and reagents used in studies described herein

| Chemical/Reagent Name | Supplier | Catalog # |
|---|---|---|
| 16% Formaldehyde (w/v), methanol-free | Thermo | 28908 |
| (PFA) | Fisher | |
| 4 [symbol] 6-diamidino-2-phenylindole | Sigma | D9 [symbol] 42 |
| (DAP [symbol] ) | | |
| 4-Hydroxy-TEMPO (4-H [symbol] ) | Sigma | 176141 |
| Acrylamide (AA) | Sigma | A9099 |
| Acrylamide/Bis 19 [symbol] 1, 40% (w/v) | Thermo | AM9022 |
| solution | Fisher | |
| Acryloyl-X, SE, 6-((acryloyl)amino) | Thermo | A20770 |
| hexanoic Acid, Succinimidyl Ester | Fisher | |
| (AcX) | | |
| Aminoallyl-dUTP solution (50 mM) | Thermo | R1101 |
| | Fisher | |
| Ammonium persulfate (APS) | Sigma | A3678 |
| Bind-silane | Sigma | GE17-1330-01 |
| BODIPY FL C [symbol] | Thermo | D3822 |
| | Fisher | |
| C [symbol] Ligase II ssDNA ligase | Lucigen | CL9025K |
| Deoxynucleotide (dNTP), 10 mM | NEB | N0447L |
| solution mix | | |
| Dideoxynucleoside triphosphate [symbol] | Roche | 03732738001 |
| (ddNTP) | | |
| DMEM | Thermo | 10569010 |
| | Fisher | |
| DMSO [symbol] Anhydrous | Thermo | D12345 |
| | Fisher | |
| D [symbol] | Roche | 37016821 |
| DPBS [symbol] 1× | Corning | 21-031-CV |
| EDTA [symbol] 0 [symbol] M solution, pH 8.0 | Thermo | 15575020 |
| | Fisher | |
| Endonuclease V | NEB | M0305S |
| Endoproteinase Ly [symbol] C | NEB | P8109S |
| Ethanolamine hydrochloride | Sigma | E6133 |
| Fetal bovine serum | Thermo | 16000036 |
| | Fisher | |
| FM 1-43 FX membrane st [symbol] n | Thermo | F [symbol] |
| | Fisher | |
| Formamide (deionized) | Thermo | AM9344 |
| | Fisher | |
| Glut [symbol] ldehyde (GA), 25% solution | Sigma | G5882 |
| Glycidyl methylacrylate (GMA) | Sigma | 151238 |
| Guanidine hydrochloride | Sigma | 50937 |
| HCR-FISH probes, amplifiers and | Molecular | N/A |
| buffers | Instruments | |
| Inosine | Sigma | [symbol] 4125 |
| Label-IT amine | Mirus Bio | MIR3900 |
| MAXpack immunostaining media kit | Active | [symbol] 5251 |
| | Motif | |
| MES [symbol] M solution, pH. 6.5 | Alfa Acsar | [symbol] 63778 |
| MiSeg reagent kit v3 | Illumina | MS-102-3003 |
| N-(3-Dimethylaminopropyl)-N [symbol] - | Sigma | 03450 |
| ethylcarbodiimide hydrochloride (EDC) | | |
| N [symbol] N [symbol] N [symbol] N [symbol] - | Sigma | T7024 |
| Tetramethylethylenediamine (TEMED) | | |
| N [symbol] N [symbol] -Methylenebisacrylamide | Sigma | M7279 |
| (BIS) | | |
| N-Hydroxysuccinimide (NHS) | Thermo | 24500 |
| | Fisher | |
| Octadecyl rhodamine B chloride (R18) | Thermo | O246 |
| | Fisher | |
| PBCV-1 DNA ligase | NEB | M0375L |
| PRS [symbol] 10× | Thermo | 70011044 |
| | Fisher | |
| PEGylated bis(sulfosuccinimidyl) | Thermo | 21582 |
| su [symbol] ate (BE(PEG)9) | Fisher | |
| Penicillin-streptomycin (10,000 U/mL) | Thermo | [symbol] 5140122 |
| | Fisher | |
| phi29 DNA polymerase | Enzymatics | P7020-HC-L |
| Proteinase K | NEB | P8107S |
| RNase A | Thermo | EN0531 |
| | Fisher | |
| RNase H | NEB | M0297L |
| RNase inhibitor | NEB | M0314S |
| Sigmacote | Sigma | SL2 |
| Sodium acrylate (SA) | Sigma | 408220 |
| Sodium bicarbonate [symbol] powder | Sigma | S6014 |
| Sodium borate [symbol] 0 [symbol] M solution, | Alfa Acsar | [symbol] 62902 |
| pH 8.5 | | |
| Sodium borohydride | Sigma | 213462 |
| Sodium chloride, [symbol] M solution | Sigma | 59222C |
| Sodium dodecyl sulfate (SDS), 20% | Sigma | 05030 |
| solution | | |
| SSC buffer, 20× | Promega | V4261 |
| Standard Tag reaction buffer (with | NEB | B9014 [symbol] |
| magnesium chloride) | | |
| SuperScript TV reverse transcriptase | Thermo | [symbol] 8090050 |
| | Fisher | |
| Terminal transferase | NEB | M03 [symbol] 5L |
| TetraSpeck microspheres, 0.5 [symbol] m | Thermo | T7281 |
| | Fisher | |
| Trimethylolpropane triglycidyl ether | Sigma | 430269 |
| (TMPTE) | | |
| Tris buffer, [symbol] M solution, pH 8.0 [symbol] | Thermo | AM9856 |
| RNase-free | Fisher | |
| Triton X-100 | Sigma | T8787 |
| Trypsin-EDTA (0.25% with phenol red) | Thermo | 25200072 |
| | Fisher | |
| Tween 20 | Sigma | P9416 |
| UltraPure DNase/RNase-free distilled | Thermo | [symbol] 0977023 |
| water | Fisher | |
| Wheat germ [symbol] (WGA) [symbol] Alexa | Thermo | W32466 |
| Fluor 647 | Fisher | |
| Zwittergent 3-10 detergent | Sigma | 693021 |

[symbol]

indicates data missing or illegible when filed

### Anchoring, Gelation, Homogenization and Expansion

Before anchoring, thick tissue samples were pre-diffused with GMA at 4° C. Fixed cells and tissue slices were then pre-incubated with 100 mM sodium bicarbonate (pH=8.5, DNase/RNase-free) 2×15 min, and incubated in designated concentration of GMA in 100 mM sodium bicarbonate for varied duration at room temperature or 37° C., dependent on detection modules and sample types (detailed anchoring conditions are provided in Table 4).

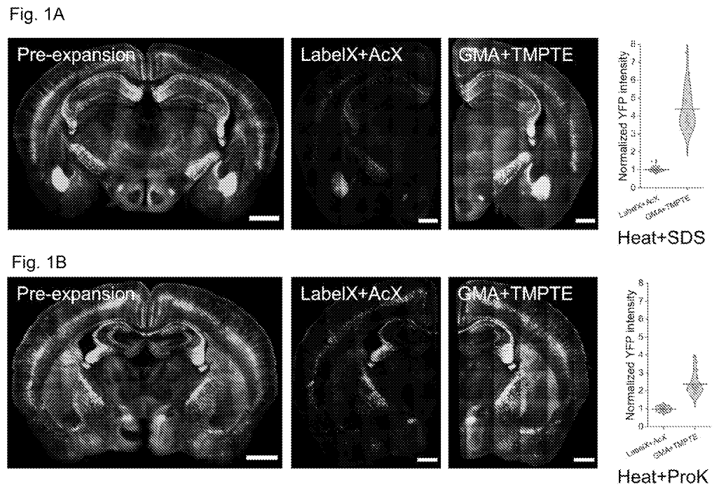

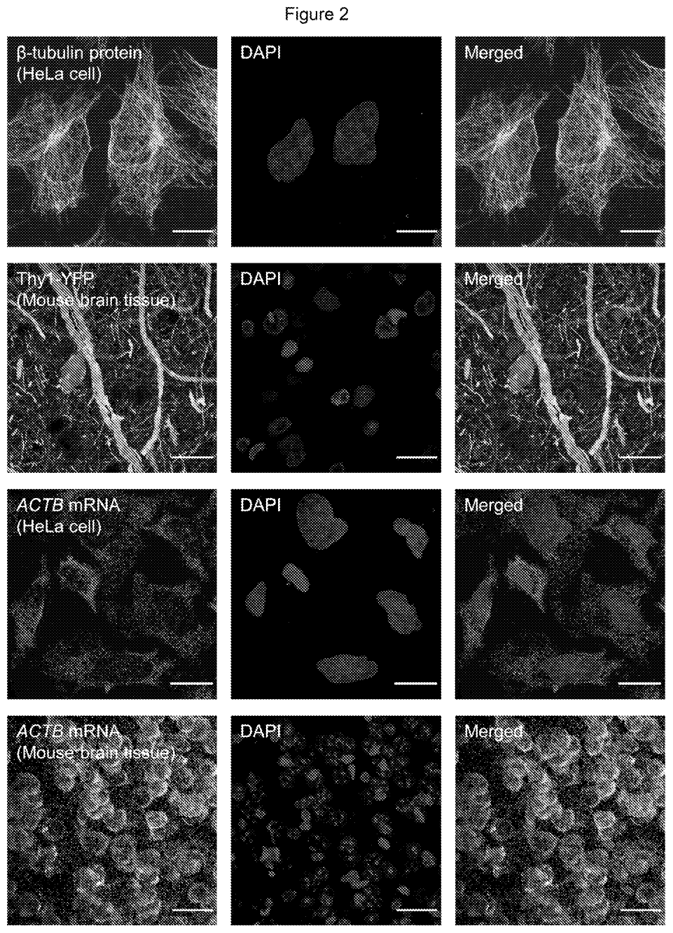

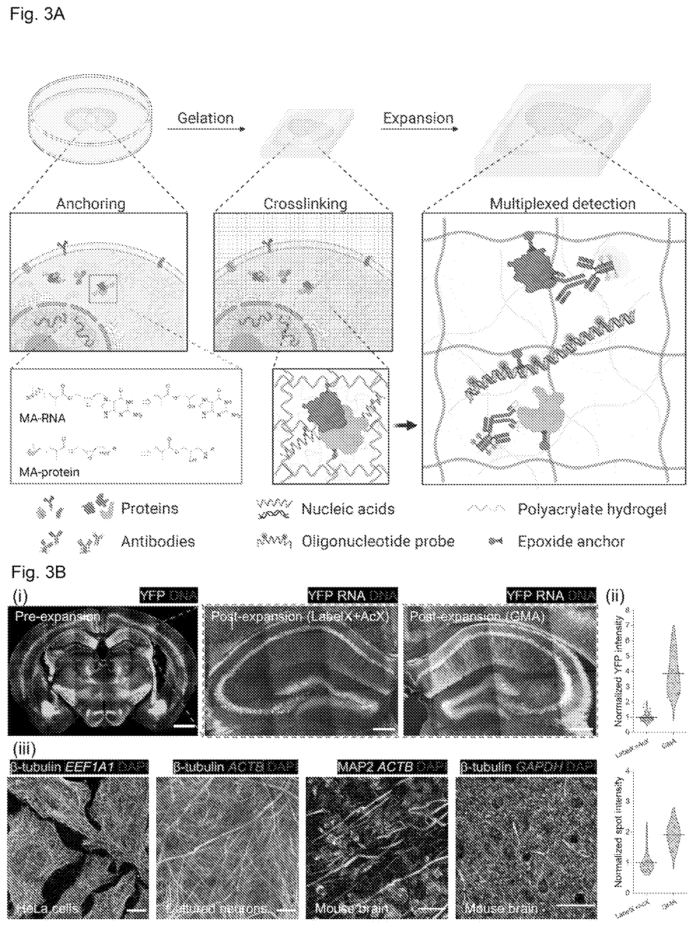

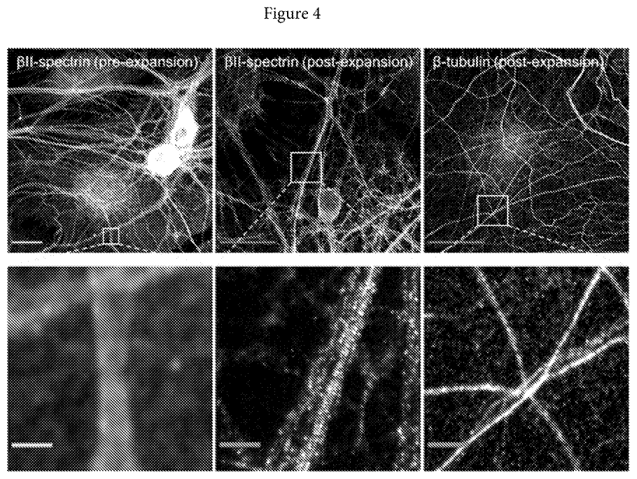

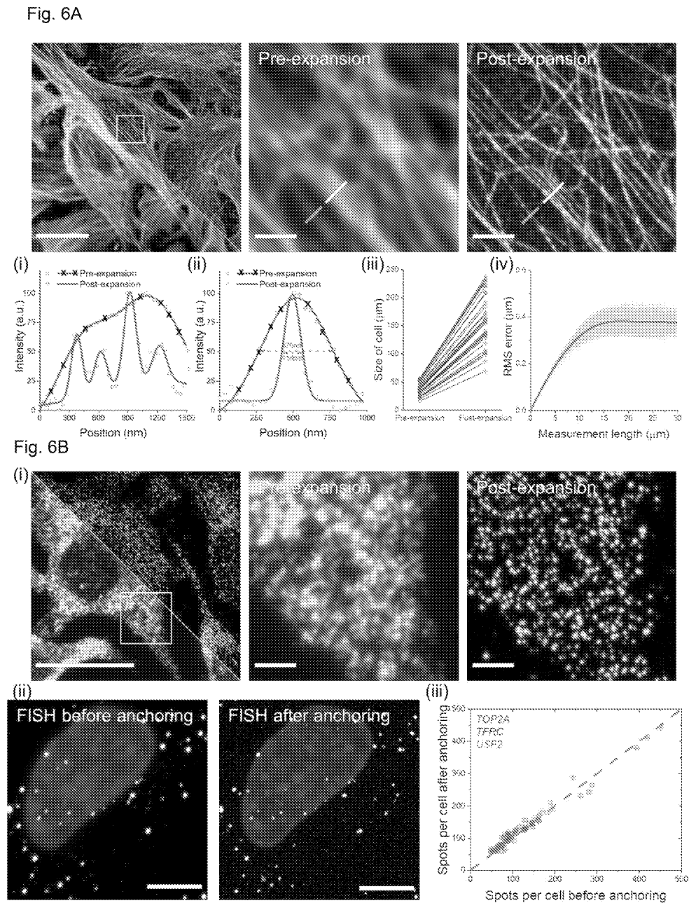

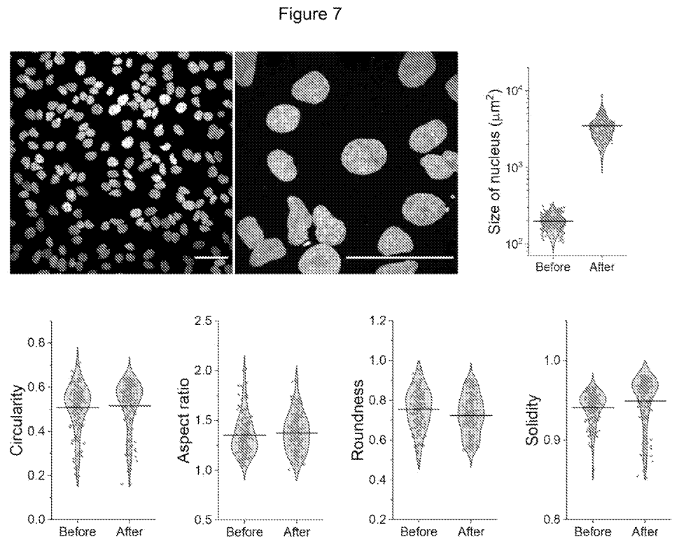

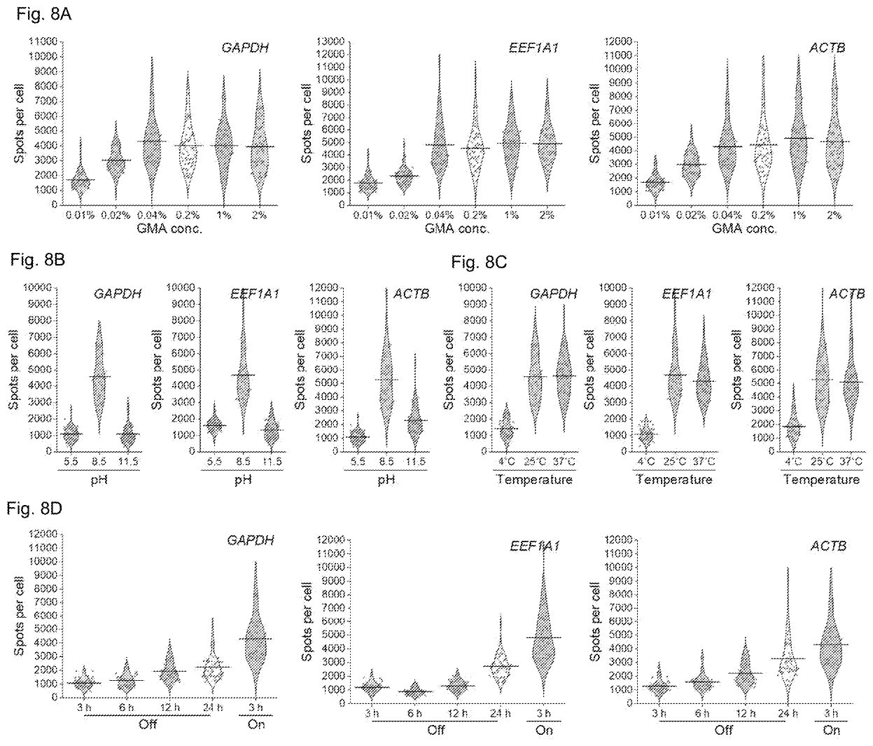

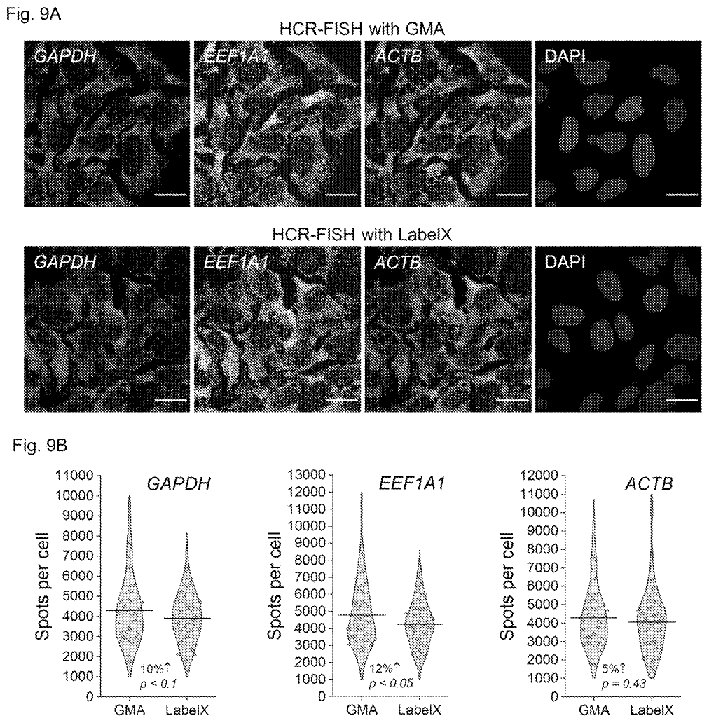

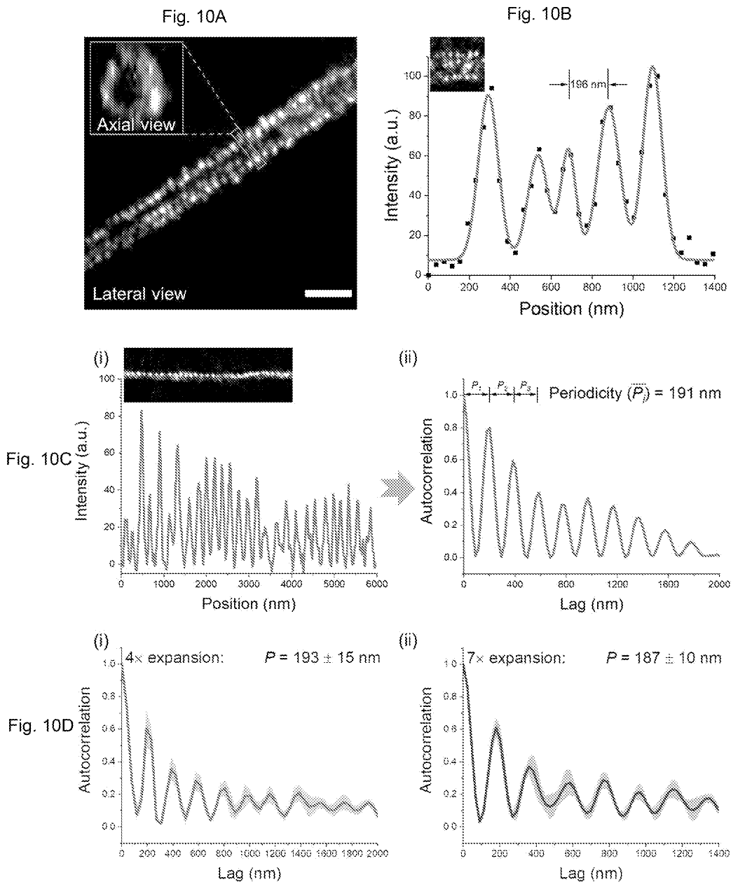

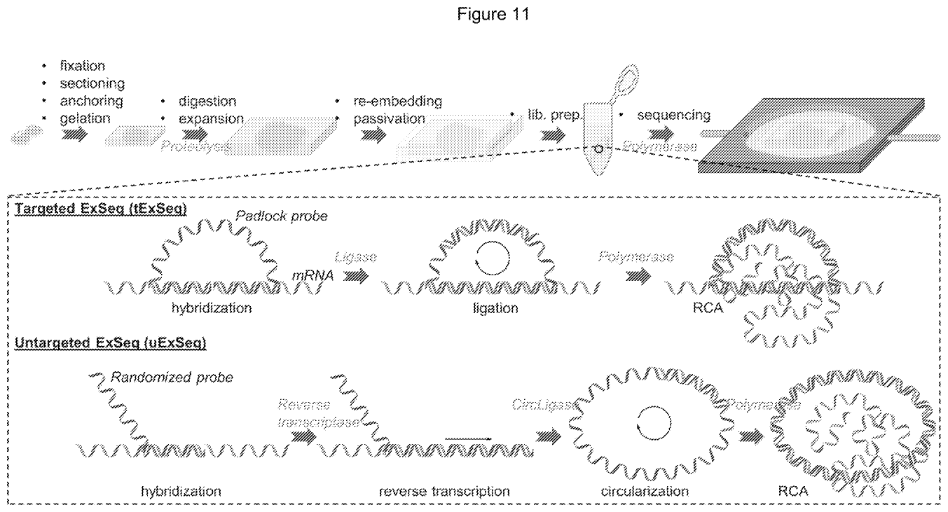

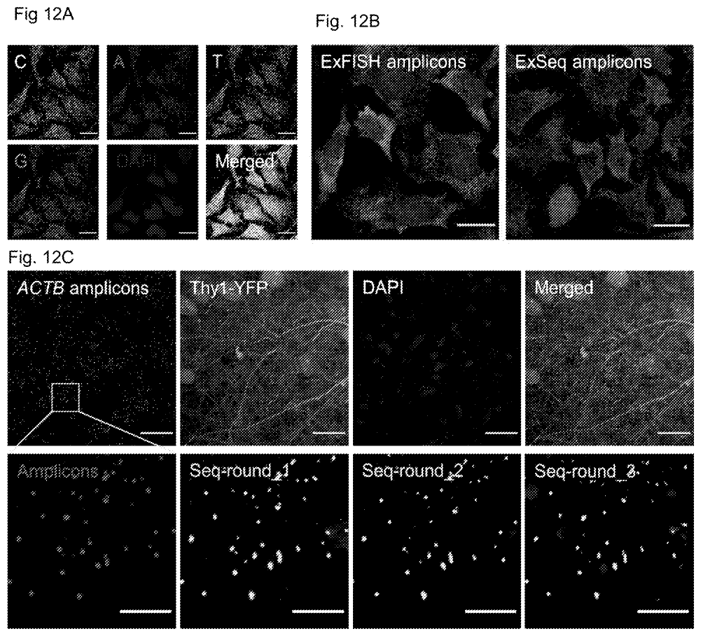

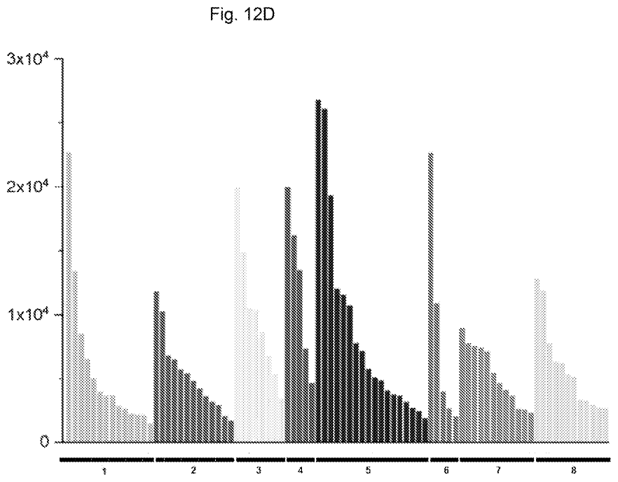

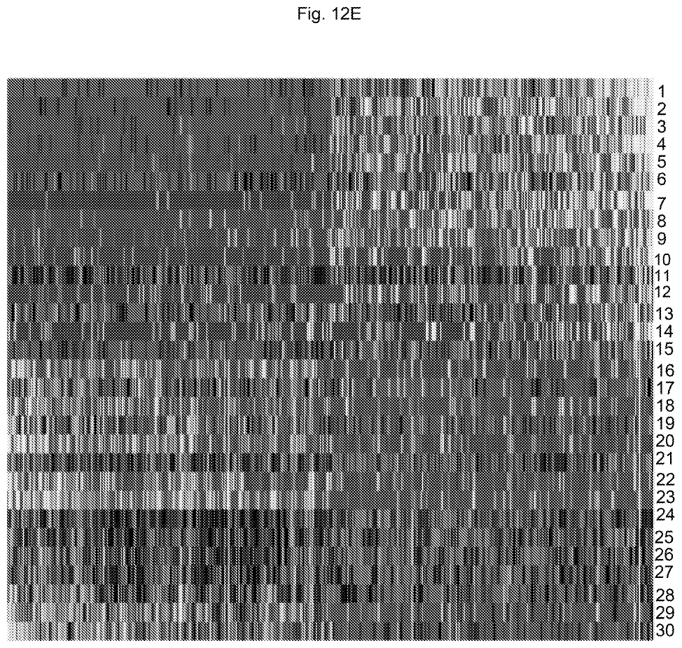

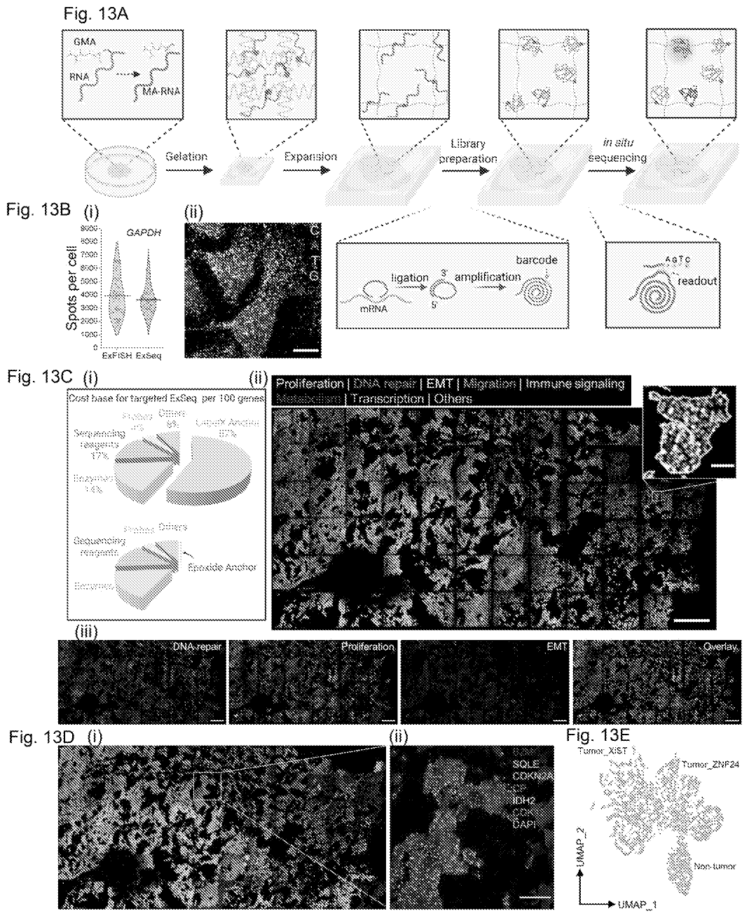

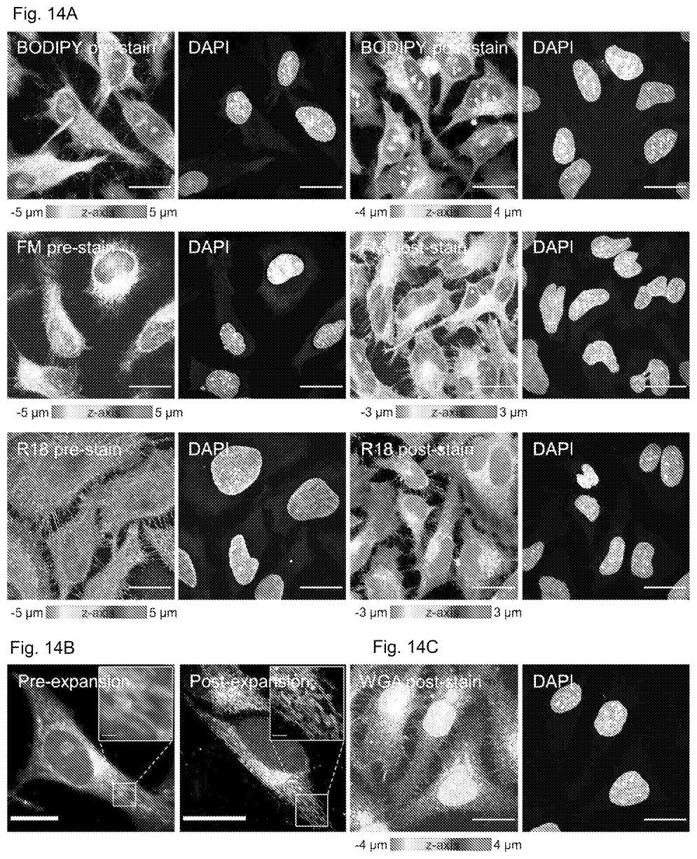

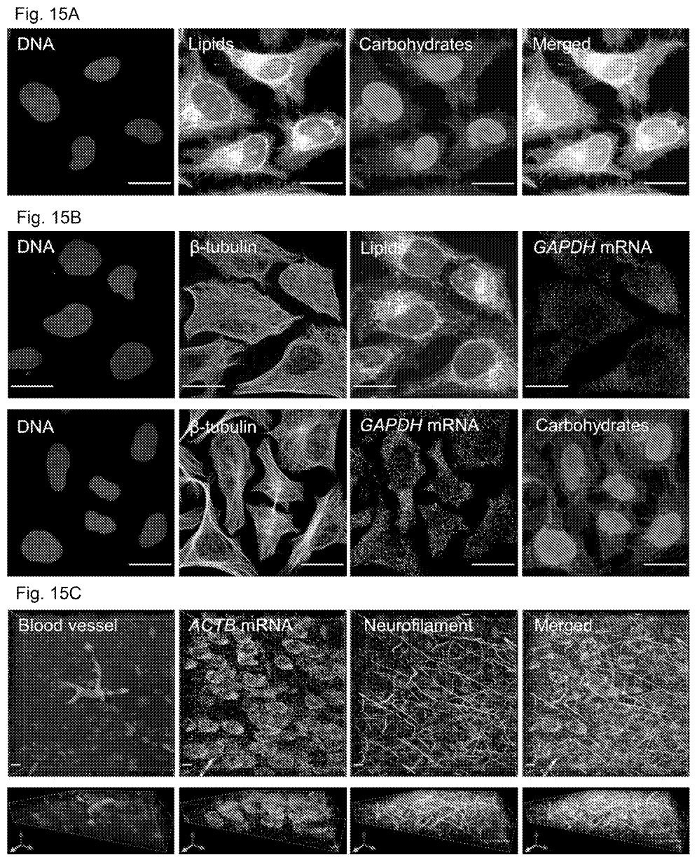

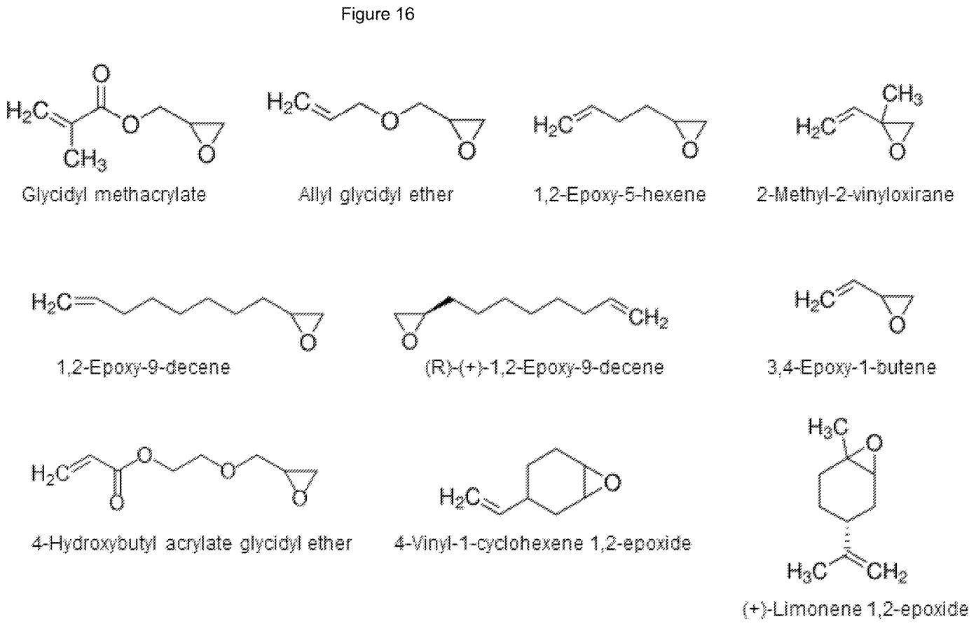

TABLE 4
anchoring conditions for various studies performed

| | Sample | | Detection |
|---|---|---|---|
| FIG. | type | Anchoring condition | module |
| FIG. 3B | Mouse | 0.1% GMA, 100 mM | post-expansion |
| (i) | brain | NaHCO [symbol] (pH 8.5), | HCR-FISH + |
| | slices* | 3-4 h at 4° C. plus 3 h | YFP signal |
| | | at 37° C. | analysis |
| FIG. 3B | Human | 0.04% GMA, 100 mM | pre-expansion |
| (iii) | HeLa cells; | NaHCO [symbol] (pH 8.5), | IF + post- |
| | Mouse | 3 h at 37° C. | expansion |
| | hippo- | | HCR-FISH |
| | campal | | |
| | neurons | | |
| FIG. 6A | Human | 0.04% GMA, 100 mM | pre-expansion |
| | HeLa cells | NaHCO [symbol] (pH 8.5), | IF |
| | | 3 h at RT | |
| FIG. 6B | Human | 0.04% GMA, 100 mM | pre-expansion |
| | HeLa cells | NaHCO [symbol] (pH 8.5), | HCR-FISH + |
| | | 3 h at RT | post-expansion |
| | | | HCR-FISH |
| FIG. 10 | Mouse | 0.04% GMA, 100 mM | pre-expansion |
| | hippo- | NaHCO [symbol] (pH 8.5), | IF |
| | campal | 3 h at RT | |
| | neurons | | |
| FIG. 13B | Human | 0.04% GMA, 100 mM | untargeted in |
| (ii) | HeLa cells | NaHCO [symbol] (pH 8.5), | situ sequencing |
| FIG. 12A | | 6 h at RT | |
| FIG. 13C | Human | 0.1% GMA, 6 h at 4° C. | targeted in situ |
| | PDX breast | (with 1× PBS, pH 7.4) | sequencing |
| | cancer | plus overnight at RT | |
| | tissues* | (with 100 mM | |
| | | NaHCO [symbol] pH 8.5) | |
| FIG. 2 | Human | 0.04% GMA, 100 mM | post-expansion |
| | HeLa | NaHCO [symbol] (pH 8.5), | IF or post- |
| | cells [symbol] | 3 h at RT [symbol] | expansion |
| | Mouse | 0.1% GMA, 100 mM | HCR-FISH |
| | brain | NaHCO [symbol] (pH 8.5), | |
| | slices* | 3-4 h at 4° C. plus 3 h | |
| | | at 37° C. (for mouse | |
| | | brain tissue) | |
| FIG. 7 | Human | 0.04% GMA, 100 mM | DAPI staining |
| | HeLa cells | NaHCO [symbol] (pH 8.5), | before imaging |
| | | 3 h at RT | |
| FIG. 8 | Human | as indicated in plots | post-expansion |
| | HeLa cells | | HCR-FISH |
| FIG. 9 | Human | 0.04% GMA, 100 mM | post-expansion |
| | HeLa cells | NaHCO [symbol] (pH 8.5), | HCR-FISH |
| | | 3 h at RT | |
| FIG. 4 | Mouse | 0.04% GMA, 100 mM | pre-expansion |
| | hippo- | NaHCO [symbol] (pH 8.5), | IF |
| | campal | 3 h at 37° C. | |
| | neurons | | |
| FIG. 1 | Mouse | 0.05% GMA + 0.05% | post-expansion |
| | brain slices | TMPTE, 6 h at 4° C. | YFP signal |
| | | (with 1× PBS, pH 7.4) | analysis |
| | | plus overnight at RT | |
| | | (with 100 mM | |
| | | NaHCO [symbol] pH 8.5) | |
| FIG. 12C | Mouse | 0.1% GMA, 6 h at 4° C. | targeted in situ |
| | brain | (with 1× PBS, pH 7.4) | sequencing |
| | slices* | plus overnight at RT | |
| | | (with 100 mM | |
| | | NaHCO [symbol] pH 8.5) | |
| FIG. | Human | 0.04% GMA, overnight | pre-expansion |
| 14A-B | HeLa cells | at 4° C. (with 1× PBS, | or post- |
| | | pH 7.4) plus 3 h at RT | expansion lipid |
| | | (with 100 mM | tag staining |
| | | NaHCO [symbol] pH 8.5) | |
| FIG. 14C | Human | 0.04% GMA, 100 mM | post-expansion |
| | HeLa cells | NaHCO [symbol] (pH 8.5), | WGA staining |
| | | 3 h at RT | |
| FIG. 15A | Human | 0.04% GMA, overnight | post-expansion |
| | HeLa cells | at 4° C. (with 1× PBS, | R18 + WGA |
| | | pH 7.4) plus 3 h at RT | staining |
| | | (with 100 mM | |
| | | NaHCO [symbol] pH 8.5) | |
| FIG. 15B | Human | 0.04% GMA, overnight | pre-expansion |
| | HeLa cells | at 4° C. (with 1× PBS, | IF + post- |
| | | pH 7.4) plus 3 h at RT | expansion |
| | | (with 100 mM | HCR-FISH + |
| | | NaHCO [symbol] pH 8.5 | post-expansion |
| | | | R18 or WGA |
| | | | staining |
| FIG. 15C | Mouse | 0.1% GMA, overnight | pre-expansion |
| | brain slices | at 4° C. (with 1× PBS, | IF + post- |
| | | pH 7.4) plus 3 h at RT | expansion |
| | | (with 100 mM | HCR-FISH + |
| | | NaHCO [symbol] pH 8.5) | post-expansion |
| | | | WGA staining |

[symbol]

indicates data missing or illegible when filed

Of particular note, the solubility of GMA is about 3% in most aqueous solutions and so the anchoring buffer has to be vigorously vortexed after addition of GMA. Considering the potential toxicity of GMA, handling of undiluted GMA needs to be done in a fume hood with sufficient ventilation. For most experiments, a 0.04% (w/v) GMA was used for anchoring. After the anchoring reaction, samples were washed with sterile DPBS three times (for samples using >0.2% GMA, washed with 70% ethanol to remove unreacted GMA before washing with DPBS). Then, standard ExM steps including gelation, digestion and expansion were conducted.

Briefly, for gelation, the monomer solution—StockX—was prepared as developed in published protocols: 8.6% (w/v) sodium acrylate (SA), 2.5% (w/v) acrylamide (AA), 0.15% (w/v) N,N′-methylenebisacrylamide (Bis), 2 M sodium chloride (NaCl), 1×PBS. Then the gelation solution was prepared by mixing StockX with 0.5% (w/v) 4-Hydroxy-TEMPO (4-HT) stock solution (required for tissue samples), 10% (w/v) N,N,N′,N′-Tetramethylethylenediamine (TEMED) stock solution, and 10% (w/v) ammonium persulfate (APS) stock solution at 47:1:1:1 ratio on a 4° C. cold block, and diffused into the sample at 4° C. for 30 min. #0 coverslips were used as spacers to cast the gel with thickness around 100 μm. Next, the chamber containing the tissue with infiltrated gelation solution was transferred to a sealed Tupperware for free-radical initiated polymerization at 37° C. For the modified 7× expansion protocol, the following monomer solution was used; 17.5% SA, 5% AA, 0.015% Bis, 2 M NaCl, 1×PBS, and mixed with 10% TEMED and APS at 198:1:1 ratio. To reduce the gel attachment to glass surfaces, the glassware can be briefly rinsed with Sigmacote reagent before use.

For most experiments, the standard proteinase K (proK) based digestion method was performed with the buffer containing 8 U/ml proK, 0.5% (w/v) Triton N-100, 1 mM EDTA, 50 mM Tris-HCl buffer (pH=8), and 2 M NaCl. The gelled samples were digested at 37° C. overnight.

For the comparison experiments of LabelX/AcX and epoxides in preservation of proteins under high heat treatments (FIG. 1), two homogenization methods were tested. In the autoclave-based denaturation, gels were incubated with the denaturation buffer containing 200 mM SDS, 200 mM NaCl, and 50 mM Tris base (pH=9) at 95° C. for 1 hour, followed by incubation at 37° C. overnight and washing with 0.2% TritonX-100 in fresh PBS to remove residual SDS before expansion. In the proK based rapid digestion, gels were digested with 8 U/ml proK, 0.8 M guanidine hydrochloride, 0.5% (w/v) Triton X-100, 1 mM EDTA, 50 mM Tris-HCl buffer (pH=8), and 2 M NaCl at 60° C. for 2 hours, followed by incubation with fresh digestion buffer at 37° C. overnight before expansion.

For most post-expansion antibody staining and experiments involving WGA staining, gelled samples were digested with 50-100 μg/mL endoproteinase LysC in 1 mM EDTA 50 mM Tris-HCl (pH=8) and 0.1 M NaCl at 37° C. overnight (for cells) or 2-3 days (for tissues). For post-expansion antibody staining targeting Thy1-YFP in mouse brain tissues (FIG. 2), the heat-based SDS denaturation was applied.

After homogenization, the gelled samples were rinsed 3 times with fresh PBS, followed by expansion with ion-free, ultrapure water (3×15 min for cells, 3×30 min for tissues). To expand LysC-digested tissues, serial incubation with decreasing PBS (1×, 0.5×, 0.1×, 30 min each) or NaCl solutions (1M, 0.5M, 0.1M, 30 min each) was conducted before expansion with water.

After 2 hours, the gelled sample was removed from the chamber, trimmed to proper size and then immersed in digestion butler. Different sample homogenization methods were applied as specified below.

### Pre-Expansion Antibody Staining

To assess the potential sample distortion during the GMA-based uniExM procedure and the improvement on imaging resolution, pre-expansion antibody staining was performed. Primary antibodies against β-tubulin, MAP2, neurofilament, GFP, and βII-spectrin were used to stain predetermined structures in different samples. In brief, samples were fixed with 3% PFA/0.1% GA (for microtubule and spectrin preservation) or 4% PFA, followed by processing with 0.1% sodium borohydride and 100 mM glycine to quench unreacted fixative residuals. MAXblock medium was used for blocking for 1 hour and then 5 μg/mL primary antibody diluted in MAXbinding medium was incubated with the sample at 4° C. overnight (or 37° C. for 2 hours). Next day, 5 μg/mL fluorescently labelled secondary antibody was used at room temperature for 1 hour. After completely washing out unbound antibodies, samples were proceeded with the anchoring and expansion steps.

### Post-Expansion Antibody Staining

For post-expansion antibody staining, gelled specimens were digested with the milder enzyme LysC and expanded. Then the samples were incubated with 5 μg/mL primary antibody diluted in MAXbind staining medium at 4° C. overnight (or 37′C for 2 hours) and washed with MAXwash medium for four times. 5 μg/mL fluorescently labelled secondary antibody was incubated with the sample to develop signals before DAPI counterstaining and expansion.

### Staining for Lipids and Carbohydrates

R18, FM and BODIPY were all tested for pre- and post-expansion staining. The working concentration for these dyes was chosen to be 10 μg/mL (diluted with fresh PBS). For pre-expansion staining, lipid tags were introduced right before the gelation step in which samples were stained for 1-2 hours at room temperature. Then the samples were anchored with GMA at 4° C. overnight, followed by incubation at room temperature for another 3 hours, digestion (proK-based) and expansion. For post-expansion staining, samples were fixed with 3% PFA/0.1% GA, and then anchored with GMA. The samples were digested with proK or LysC (for WGA staining), followed by expansion. After expansion, the samples were stained with antibodies or HCR-FISH first (if any), and then with lipid tags for 1-2 hours at room temperature. The residual dyes were washed off with 1% Zwittergent in DPBS.

### Expansion Fluorescence In Situ Hybridization (ExFISH)

For most ExFISH experiments, expanded gels were re-embedded with another layer of 3% AA-based (plus 0.15% BIS, 5 mM Tris base pH 9, and 0.05% APS/TEMED), non-expandable gel to maintain rigidity. With the re-embedding step, the expansion factor would decrease to ˜3.2 compared to the original expansion factor of ˜4.2. Two stacked #1.5 coverslips were usually used as the spacers for re-embedding. The HCR-FISH probes and reagents were purchased from Molecular Instruments, Inc. In general, the gel was incubated with hybridization buffer at room temperature for 30 min, and then with 1:500 diluted gene-specific probe (8 nM total final probe concentration) set at 37° C. overnight. Next day, the gel was washed with HCR washing buffer at 37° C. for 4×30 min and with 5×SSCT buffer (5×SSC buffer containing 0.1% Tween 20) at RT for 4×15 min, followed by incubation with 1:200 diluted, fluorescently labelled HCR hairpin amplifiers at room temperature overnight. Lastly, the gel was washed with 5×SSCT for 4×20 min and counterstained with 1 μg/mL DAPI. To characterize RNA capture efficiency by GMA, HCR-FISH against the same genes was performed in the same sample before and after anchoring. Before anchoring, HCR-FISH was done in HeLa cells and then the hybridized probes were removed with 80% formamide. Then, ExFISH after GMA-based uniExM was done with the same sample, where the same cells were imaged in both conditions. Transcripts in single cells were counted using MATLAB scripts as developed before [Cui, Y. et al., Nucleic Acids Res (2018), Vol. 46, e7; Cui, Y. et al, Nano Lett (2019). Vol. 19, 1990-1997].

### Expansion Sequencing (ExSeq)

The detailed protocol for ExSeq was published previously and involves a multi-day procedure [Alon, S. et al., Science (2021), Vol. 371(6528)]. 87 target genes were chosen based on the top most variable genes between cancer clones in the SA501 PDX line [Campbell, K. R. et al., Genome Biol (2019), Vol. 20(54) 1-12]. In brief, a re-embedded gel was passivated with 2 M ethanolamine, 150 mM EDC and NHS. Then the passivated gel was subjected to targeted ExSeq (tExSeq) or untargeted ExSeq (uExSeq). For tExSeq, padlock probes targeting specific mRNAs (in general, 12-16 probes per gene and 100 nM per padlock probe diluted in 2×SSC containing 20% formamide) were used to hybridize with the sample at 37° C. overnight. Then the unhybridized probes were completely washed off and the sample was treated with 1.25 U/μL PBCV-1 DNA ligase at 37° C. overnight, followed by inactivation at 60° C. for 20 min. Next, the successfully ligated padlock probes were rolling circle amplified with 1 U/μL phi29 DNA polymerase. As all padlock probes targeting the same gene bear a predetermined barcode, the identity of the mRNA can be read out by commercially available sequencing reagents (e.g., the illumina MiSeq kit). In comparison, uExSeq utilizes randomized 8N oligonucleotide probes to hybridize with any potential RNA targets without prior sequence knowledge. After that, reverse transcription was performed in situ with 10 U/μL SSIV reverse transcriptase to generate cDNAs containing inosine. The cDNAs were later segmented to proper sizes with endonuclease V and circularized with 3 U/μL CircLigase. Then the target mRNAs were digested away with RNase H. Such circularized cDNAs were subjected to rolling circle amplification and sequencing readout. For detailed working mechanism and protocols of tExSeq and uExSeq, see previous work [Alon, S. et al., Science (2021), Vol. 371(6528)].

The sequencing-by-synthesis chemistry was adapted for in situ 7-base readout using the Illumina MiSeq v3 kit with a modified protocol. To help the registration process, the re-embedded gel sample was adherent to bind-silane (1:250 diluted in 80% ethanol) processed glass surface with the same re-embedding monomer solution containing 1:100 diluted TetraSpeck microspheres. Before sequencing, the sample was first treated with 400 U/mL, terminal transferase and 50 μM ddNTP to block nonspecifically exposed 3′ ends in DNA, and then hybridized with 2.5 μM sequencing primer (5′-tctcgggaacgctgaagacggc-3; SEQ ID NO: 1) in 4×SSC at 37° C. for 1 hour. After 3×10 min washing with fresh 4×SSC, the sample was incubated with the PR2 incorporation buffer (part of the MiSeq kit) for 2×15 min. Then the sample was pre-incubated with 0.5× incorporation mix buffer (IMT of the MiSeq kit) supplemented with 1× Taq fact polymerase buffer and 2.5 mM magnesium chloride at RT for 2×15 min. Then the sample was incubated with 0.5×IMT at 50° C. for 10 min for one base elongation. After the elongation reaction, the sample was washed with PR2 containing 2% Zwittergent at 50° C. for 2×15 min followed by additional washing with PR2 at RT for 2×15 min. Next, the sample was immersed in imaging buffer (SRE of the MiSeq kit) and subjected to imaging (elaborated in the following section). After imaging, the sample was briefly washed with PR2 at RT for 2×10 min. Then the sample was incubated with cleavage solution (EMS of the MiSeq kit) at 37° C. for 3×15 min. Lastly, the sample was washed with PR2 at 37° C. for 2×15 min and at RT for 2×15 min, and then started with the next round of elongation process.

### Data Analysis for ExSeq

Data analysis fur the sequenced PDX sample followed our established ExSeq processing pipeline (available at: github.com/dgoodwin208/ExSeqProcessing). For the 87-gene probe set, a 7-base barcoding strategy with error correction capacity was adopted. Upon microscopic readout, the raw image files were stored in 16-bit HDF5 format and subjected to color correction, registration, segmentation, basecalling and alignment as done in our previous work, [Alon, S. et al., Science (2021), Vol. 371(6528)]. and then performed manual cell segmentation in 2D according to a max-Z projection of the DAPI staining channel using the VASTLite package (//lichtman.rc.fas.harvard.edu/vast/). In total, 793,535 unique transcripts were detected for a population of 3,339 cells (with effective lateral resolution ˜78 nm and axial resolution of ˜160 nm). For gene function annotation we refer to The Human Protein Atlas (proteinatlas.org) or The Human Gene Database (genecards.org). The spatial maps of single transcripts or functional gene groups were generated with MATLAB scripts (for coordinates extraction) and ImageJ packages (for visualization).

For biostatistics analysis the R toolkit Seurat 4 was utilized. Before analysis, the dataset was further pruned based on the counts per cell values, where cells with less than 50 counts or more than 3000 counts were filtered out and the result was 2,732 cells. Then the counts per cell were normalized by the median value from all the cells and performed a log transformation. To identify cell clusters, both unsupervised and semi-supervised approaches were applied. In unsupervised clustering, PCA suggested a majority of cells could be classified to two clusters using the expression profile of 30 genes. With that, a studies were performed to visualize these two cell groups based on the relative expression of these 30 genes (by correlating the percentages of the top and bottom 15 genes in each single cell with the color channel intensities of an RGB composite image). Therefore, for every cell, its closeness to a particular group rather than an arbitrary binary classification was presented so that the transitional status of different tumor clones may be preserved. The gene list used for semi-supervised clustering was selected based on RNA-seq data, in which the 15 most up-regulated genes (RFP146, DDX24, OAZ2, ZNF24, TXNL1, IDH2, SEPT4, CDCA7, CP, RAD21, WDR61, RBP1, COX5A, HSPE1, IER3IP1) and 15 most down-regulated genes (XIST, CD44, FBXO32, LGALS1, ARC, HLA-A, HLA-C, S100A11, CTSV, SLC25A6, ANXA1, ARHGDIB, SQLE, B2M, NDUFS5) in the SA501 PDX model were applied for the initial dimension reduction. Afterwards, the major cell clusters were presented with uniform manifold approximation and projection (UMAP).

### Imaging and Image Analysis

All imaging experiments were performed on a spinning disk confocal microscope (Andor Dragonfly) equipped with a Zyla sCMOS 4.2 plus camera (pixel size 6.5 μm) or a CSU-W1 SoRa super-resolution spinning disk confocal microscope (Nikon). Six main lasers on Dragonfly were used: 405 nm (100 mW), 488 nm (150 mW), 561 nm (150 mW), 594 nm (100 MW), 637 nm (140 mW) and 685 nm (40 mW). For whole-brain tiled scanning, a 10× objective lens was used (FIG. 3B and FIG. 4). For long-time imaging, a Nikon CFI Apochromat LWD Lambda S 40XC water immersion objective lens (working distance 0.6 mm, 1.15) was used together with Zeiss Immersol (Refractive Index .1.3339). For ExSeq using Illumina Miseq reagents, the following bandpass filters were used: 705-845 nm for base “C” channel, 663-737 nm for base “A” channel, 575-590 nm for base “T” channel: 500-550 nm for base “G” channel. All channels used 200 ms as the exposure time except that base “G” used 400 ms exposure time. For the sequenced cancer tissue, a total of 12×6 fields of view (FOV dimension in pre-expansion units: 104×104×62.5 um³) were captured.

For characterization of expansion in uniExM, HeLa cells stained with β-tubulin antibody and DAPI were used. The size of cells was determined by measuring the distance between two furthest apart microtubule points, and this measurement was performed on the same cells pre- and post-expansion. In parallel, the area and shape descriptors of cell nuclei were measured with ImageJ. The following four parameters were obtained;

$$Circularity = \frac{[ Area ]}{{[ Perimeter ]}^{2}} , Aspect ratio = \frac{[ Major axis ]}{[ Minor axis ]} , Roundness = 4 \times \frac{[ Area ]}{π \times {[ Major axis ]}^{2}} , Solidity = \frac{[ Area ]}{[ Convex area ]} .$$

Quantification of expansion errors was performed as previously described [Chen, F. et al., Science (2015), Vol. 347, 543-548; Tillberg, P. W. et al. Nat Biotechnol (2016), Vol. 34(9), 987-992]. In brief, HeLa cells were stained with β-tubulin antibody pre-expansion. The same cells were imaged both pre- and post-expansion, where the pre-expansion images were taken with a Nikon SoRa super-resolution microscope (similar spatial resolution to super-resolution SIM). The obtained images were first histogram normalized and deconvolved in imageJ. Then non-rigid registration was performed using B-spline grids to capture potential non-uniformities between images,

For periodicity analysis of βII-spectrin, cultured neurons were stained pre-expansion. Then the cells were expanded and imaged. Segments of neuronal processes with more than 10 spectrin signal clusters were selected and relevant fluorescence profiles were extracted. The fluorescence traces in space were scaled back to the pre-expansion level and autocorrelation was performed in OriginLab software. From the obtained autocorrelation curve, periodicity was calculated by averaging the distances of the first four adjacent peaks.

### Results

### Epoxide is a Multifunctional Anchor

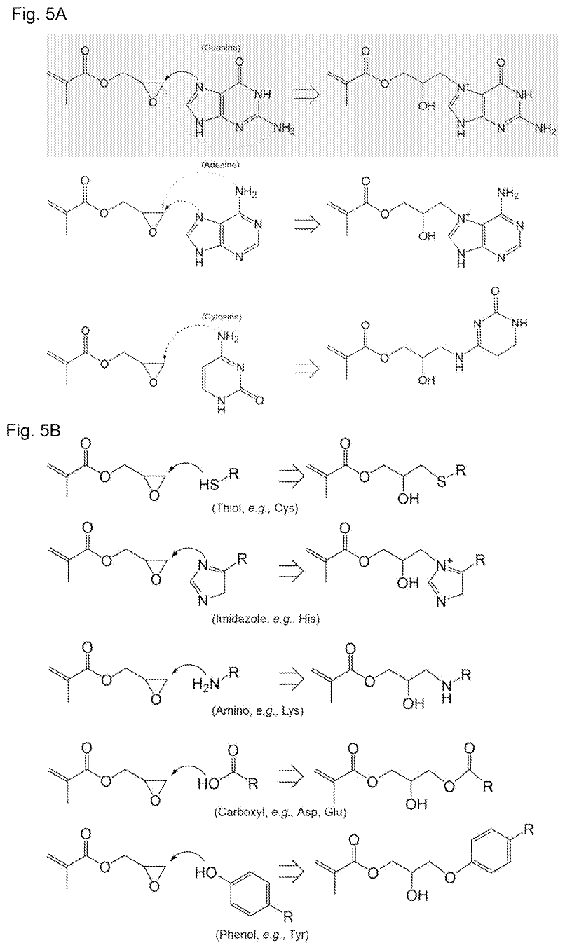

The ring-opening process of epoxides is a nucleophilic substitution reaction and could follow two pathways: the S_{N}1-like reaction under acidic condition or S_{N}2 reaction under basic condition, making the anchoring reaction pH sensitive. Acidic solutions are able to protonate epoxides and open the high-tension three-atom ring directly, resulting in rapid conjugation with weak nucleophiles such as water and alcohol. However, acids could also protonate a majority of intracellular nucleophiles such as amine groups and N7-guanine, therefore inhibiting the anchoring of these critical biomolecules. In part on this basis, a slightly basic system (pH=8.5, buffered by 100 mM sodium bicarbonate) was utilized in certain embodiments to permit epoxides to react with intracellular nucleophiles of biological importance, including but not limited to cysteine, histidine, lysine, glutamic acid, tyrosine, guanine, and some lipids and carbohydrates (FIG. 5) [Chen, G. et al., J Am Chem Soc (2003), Vol. 125, 8130-8133; Richter, S. et al., Nucleic Acids Res (2003), Vol. 31, 5149-5156; and Park, Y. G. et al., Nat Biotechnol (2018), Vol. 10(1038)]. In spite of some shared similarity to the aziridinium-dependent alkylation by LabelX, anchoring by GMA is more controllable and versatile in reaction. The significantly smaller size of GMA facilitates better diffusion and penetration into complex tissue specimens, conducive to uniform anchoring and isotropic expansion. A high-level comparison of LabelX and GMA with regards to key properties in application is summarized in Table 5.

TABLE 5
Comparison between LabelX and GMA for key EXM-related parameters.

| | LabelX (Label-IT + AcX) | GMA |
|---|---|---|
| Chemical formula | 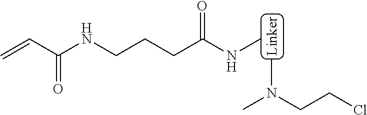 | 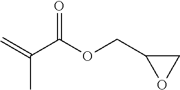 |
| 3D structure | 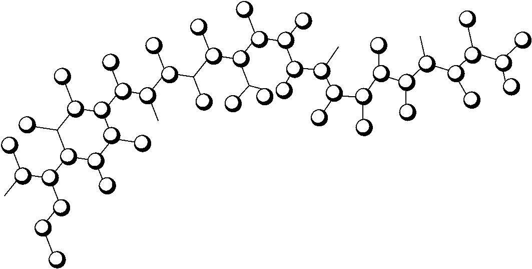 [SMILES: CCCC(C(C)C)C1C(C)C(C)C(C(C)C(C)C(C)C(C)C(C)C(C(C)C)C(C)C(C)C(C)C(C)C(C)C(C)C(C)C(C)C(C)C(C)C(C)C(C)C)C(C)C1C] | 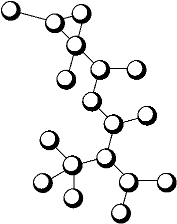 |
| | LabelX | GMA |
| Molecular weight | ~500 | ~140 |
| Reactive moiety | Aziridinium | Epoxide |
| Diffusion rate | Moderate as in original protocol | >50% faster than LabelX |
| Primary anchoring | Nucleic acids (via Guanine-N7) | Major intracellular nucleophiles, |
| substrates | | including nucleic acids, proteins |
| | | and beyond |
| Anchoring time | >10 h (at 37° C., pH 7.7) | 3 h (at RT or 37° C., pH 8.5) |
| Working conc. | 0.02-0.1% (w/v) | 0.02-0.1% (w/v) |
| Stability | 6 months at −20° C. | >2 years at 2-8° C. |
| Cost per unit | Label-IT amine: $7,000/mg | $0.0003/mg |
| | AcX: $30/mg | |
| Cost per sample | ~$170 (200-250 μL anchoring | ~$0.000075 (400-500 μL |
| (e.g., for a 50 μm | reaction volume) | anchoring reaction volume) |
| mouse brain slice) | | |
| Total cost for one | ~$190 (2-plex proExM + ExFISH) | ~$20 (2-plex proExM + ExFISH) |
| standard expt. on | ~$200 (4-plex ExFISH) | ~$30 (4-plex ExFISH) |
| cm-sized tissues | ~$290 (100-plex ExSeq) | ~$120 (100-plex ExSeq) |

Considering the reactive versatility of epoxides, we tested the performance of uniExM in preservation of biomolecules was tested using published protocols. First and foremost, studies described herein confirmed that uniExM is compatible with both proExM and ExFISH (FIG. 2), where protein and RNA molecules could be simultaneously retained and resolved with higher resolution under a common confocal microscope (FIG. 3B(i). Specifically, 0.04% (w/v) GMA enabled a much better preservation of proteins and RNAs compared with the established method using 0.02% (w/v) LabelX plus additional 0.005% (w/v) AcX, [Chen, F. et al. Nat Methods (2016): Vol. 13(8), 679-684] supporting its high efficiency of reaction. In mouse brain tissues expressing Thy1-YFP, after digestion and expansion GMA helped retain a high level of YFP fluorescence in addition to ExFISH signals, whereas LabelX plus AcX preserved relatively modest YFP signals. On average, the YFP intensity in GMA treated tissues was about 4 times stronger than that in LabelX/AcX treated tissues, and the single-spot intensity of HCR-FISH was 2 times stronger in GMA treated tissues (FIG. 3B(ii), likely due to the high stability, superior tissue penetration, multivalent anchoring and more homogeneous expansion by GMA. In concordance with results described herein, it has been reported that better anchoring could indeed improve signals of HCR-FISH after expansion [Wang, Y. et al, Cell (2021), Vol. 184, 6361-6377]. By unifying proExM and ExFISH with a single epoxide anchor, co-detection of representative protein and mRNA targets in HeLa cells, cultured mouse hippocampal neurons and sectioned mouse brain tissues [FIG. 3B(iii)] has confirmed efficacy of embodiments of methods of the invention and use of epoxide as an anchor. Embodiments comprising this significantly improved protocol are referred to herein as “unified Expansion Microscopy” (uniExM).

Characterization for uniExM

The performance of uniExM was quantitatively evaluated in several key criteria. The resolution improvement by uniExM was determined to assist in resolving line structures inside single cells [FIG. 6A(i)], in HeLa cells stained with β-tubulin antibody, uniExM enabled closely adjacent microtubules to be distinguished with much enhanced spatial resolution from a typical confocal microscope [FIG. 6A(ii and iii)]. The expansion factor was calculated to be 4.2-4.4 using 0.04-0.1% GMA [FIG. 6A(iv)] and the expansion error rate was similar to previously published results (˜1.3% over a 30-100 μm measurement scale, benchmarking against Nikon SoRa super-resolution microscopy) [FIG. 6A(v)]. In addition, the morphology and geometry of important cellular structures such as nucleus were reliably maintained after expansion (FIG. 7).

It was determined that uniExM also facilitated retention of RNA molecules with high efficiency and accuracy. Studies were performed to systematically examine different concentrations of GMA, varied reaction pH and temperature in anchoring three highly expressed genes (GAPDH, EEF1A1, ACTB, 1000-10000 transcripts per single HeLa cell) (FIG. 8). Without expansion, precise counting of these mRNAs in single cells is challenging due to the over-crowded transcripts. In comparison, the de-crowding effect brought about by expansion enabled single-cell quantification of transcripts [FIG. 6B(i)]. An optimal reaction condition was determined to be 0.04-0.1% GMA at pH 8.5 (100 mM NaHCO₃). In the pH tests, over acidic or basic pH resulted in insufficient anchoring. Under acidic pH (<6), nucleophilic sites such as N7-guanine are protonated, blocking their reaction with epoxides. Moreover, acidic pH could lead to nonspecific ring-opening of epoxide and non-enzymatic depurination, detrimental to the preservation of nucleic acids. On the other hand, under basic pH (>11), RNA was subject to deleterious hydrolysis, giving rise to substantial loss in the HCR-FISH readout (FIG. 8B). In the temperature tests, it was determined that room temperature (different from 37° C. required by LabelX) was compatible with the epoxide anchoring reaction (FIG. 8C), hence beneficial for the preservation of heat-sensitive biomolecules (e.g., membranes) over long term. Moreover, it was determined that the anchoring reaction could be efficiently controlled by temperature and pH together. In a non-limiting example, at 4° C. and neutral pH, the anchoring rate could be suppressed by more than 50% after 12 h incubation, while it can be rapidly recovered to 100% by additional 3 h incubation at 25° C. and pH 8.5 (FIG. 8C). This property makes GMA an ideal anchor for processing thick tissue samples where a uniform distribution is needed or otherwise immature anchoring would consume a large amount of GMA molecules at the periphery of the specimen, causing imbalanced retention of molecules.

To characterize anchoring efficacy, three moderately expressed genes (TOP2A, TFRC, USF2, 50-500 transcripts per single HeLa cell) were quantified and compared with HCR-FISH before and after GMA anchoring. The results support that uniExM could effectively preserve all the target RNA molecules and the retention efficiency reached almost 100% when using HCR-FISH results as the benchmark [FIG. 6B(ii and iii)]. After determining the optimal GMA anchoring condition, head-to-head comparison between GMA and LabelX was performed. The results suggest that GMA was not inferior to, or was slightly better than LabelX in preservation of highly expressed mRNA targets (FIG. 9). Thanks to its significant cost advantage over LabelX, GMA is an ideal alternative to LabelX and provides superb affordability to spatial transcriptomics researches with expansion-enabled nanometer resolution.

uniExM for Preservation of Protein Content and Ultrastructures

Studies were carried out to evaluate the performance of uniExM in preservation of ultrastructure in cells. Considering the fact that GMA is able to anchor a variety of amino acids, it was of interest to determine if that could better maintain and facilitate analysis of delicate ultrastructures in the context of expansion. To test this, βII-spectrin was selected as a target molecule due to its periodic distribution in axons as discovered by super-resolution imaging. [Xu, K. et al., Science (2013), Vol. 339, 452-456: He, J. et al,, Proc Natl Acad Sci USA (2016), Vol. 113, 6029-6034]. Mouse hippocampal neurons were dissected and cultured, and pre-expansion antibody staining was performed. Using the standard 4× expansion protocol, periodic distribution of βII-spectrin was prevalently observed in axons (FIG. 10A). The distance between two adjacent βII-spectrin spots was found to be about 190 nm and universal in different segments of axons (FIG. 10B). In the control experiments, imaging of βII-spectrin before expansion or β-tubulin after expansion show a continuous distribution (FIG. 4), which ruled out the possible expansion caused artifacts. Furthermore, the long-range periodic distribution of βII -spectrin could be quantified with autocorrelation analysis (FIG. 10C). To corroborate this spatial pattern by viewing at even higher resolution, an in-house developed 7× expansion protocol by adjusting the ratio of monomers and crosslinkers) was applied, in which a similar quasi-1D periodic structure was observed and validated by SoRa super-resolution imaging, indicating a homogeneous anchoring of biomolecules by GMA. Finally, autocorrelation analyses with multiple biological and technical repeats were performed, where the calculated periodicity values (193±15 nm for 4× expanded cells, 187±10 nm for 7× expanded cells) are consistent with the previous results obtained by super-resolution STORM or STED imaging (FIG. 10D) [Sidenstein, S. C. et al., Sci Rep (2016), Vol. 6(26725) 1-8; Martinez, F. et al. Sci Rep (2020), Vol. 10(2917) 1-11]. The application of epoxide-based uniExM could be further extended by combinatorial use of acrylate epoxides and polyepoxides. It has been demonstrated that polyepoxide enables controlled inter- and intramolecular crosslinking and therefore transforms the treated tissues being resistant to degradation under harsh conditions, such as heating [Park, Y. G. et al., Nat Biotechnol (2018), Vol. 10(1038)]. In this study GMA was combined with a polyepoxide, trimethylolpropane triglycidyl ether (TMPTE), to assess their performance in preservation of protein content after high heat-involved rapid digestion or denaturation. Studies were performed to test two tissue-processing protocols requiring heating above 50° C.: the SDS-based autoclave denaturation (95° C. for 1 h) and the proK-based enzymatic digestion (60° C. for 2 h). Compared with LabelX plus AcX, GMA plus TMPTE show substantial superiority in retention of fluorescence signals for PFA perfused and fresh frozen Thy1-YFP mouse brain tissues (an average 440% signal improvement for SDS-based denaturation and 250% signal improvement for proK-based digestion) (FIG. 1).

uniExM for Cost-Effective In Situ Sequencing

Another powerful application for expansion microscopy is Expansion Sequencing (ExSeq), which enables high-resolution targeted and untargeted mapping of transcripts. ExSeq not only de-crowds compacted RNA molecules but also frees up sample inner spaces, facilitating reagents delivery and reactions in situ. In untargeted ExSeq, linear probes containing randomized octamer sequences were hybridized with RNA targets, followed by reverse transcription to write RNA sequence information into cDNAs. The synthesized cDNAs were then circularized and underwent rolling circle amplification (RCA) (FIG. 12). Hence, in each round of sequencing readout, all the four base colors could be detected with optical imaging (FIG. 12A). The abundant signal spots proved the compatibility of aforementioned enzymatic reactions with the unit uniExM-anchored RNA molecules. Because untargeted ExSeq does not require prior knowledge about RNA sequences, it can be used to study biological processes such as alternative splicing that are inaccessible to conventional in situ technologies.

Besides untargeted ExSeq, studies were carried out that demonstrated successful use of targeted ExSeq across a number of different samples (FIG. 13A). First, the detection efficiency of uniExM-based targeted ExSeq was evaluated. In HeLa cells, padlock probes targeting GAPDH mRNAs were introduced, where successfully hybridized probes went through ligation and RCA. The amplicons were counted in each individual cell and compared to the quantification results using ExFISH (FIG. 12B). The copy number values in single cells obtained by these two methods were highly concordant (FIG. 13B, i), supporting the use of GMA in quantitative ExSeq analysts. In addition, these amplicons could be subsequently sequenced using Illumina MiSeq reagents with a high signal-to-noise ratio (SNR) (FIG. 13B, ii). Next, padlock probes targeting ACTB mRNAs were used to test the stability of uniExM-based ExSeq amplicons across multiple rounds of sequencing in mouse brain tissues. The padlock probes contain repetitive “T” bases in their barcode region and so the amplicons should emit the same fluorescence signals in each round of sequencing. The amplicons were first examined with a universal detection probe to generate a reference image, then followed by three consecutive rounds of sequencing (FIG. 12C). It can be seen that amplicons were consistently detected in each round, indicating that the uniExM-based ExSeq is compatible with the full cycle of the sequence-by-synthesis (SBS) chemistry (i.e., elongation, detection and cleavage). Moreover, the Thy1-YFP fluorescence was preserved in uniExM tissues, providing opportunities to analyze transcriptomics in the context of morphological traits without additional staining as required by the original ExSeq protocol. Taken together, the results provided evidence that the uniExM-based anchoring could greatly preserve nucleic acids and was suitable for spatial transcriptomics studies.

Due to the improved performance of uniExM-based ExSeq, studies were carried out that included 7-round SBS to profile 87 genes in breast cancer PDX tissues, whereas the original LabelX-based ExSeq was limited to 4-round SBS. Importantly, uniExM significantly reduced the cost base for ExSeq as the anchoring reagent contributes to more than half of the entire cost in the original protocol, which now is nearly zero (FIG. 13C, i). In a 1.24×0.62 mm² region of interest, 793,535 raw reads (colored by function annotations) and 3.339 effective cells were detected (FIG. 13C, ii). A notable challenge for analyzing this PDX samples is that a majority of cancer cells of a relatively small size (<10-20 μm) are densely packed, making quantitative analysis using other spatial technologies (without expansion) extremely difficult. ExSeq enables high-resolution mapping of transcripts at the single-cell level and provides tremendous avenues for us to explore the underlying biology. The reads were first grouped based on their functional annotation and cell-to-cell differences in space were visualized (FIG. 12D). As shown in FIG. 13C, iii, transcripts from three functional groups were presented as 8-bit “RGB” images where the color intensity positively correlates to the expression level in each individual cell (i.e., percentage of the transcript reads of a specific group in the total reads). Upon overlay, the composite image clearly reveals a drastic heterogeneity of this cancer tissue, in which varied functions of “DNA repair”, “proliferation” and “EMT” exhibit distinct “hot spots” of spatial expression. Next, principal component analysis (PCA) was applied and from two top principal components (PC) two distinct cell groups were identified (FIG. 12E). Based on the expression level of these 30 PC genes, a color code was arbitrarily assigned to each individual cell (FIG. 13D, i). The summed counts of the top 15 genes and bottom 15 genes in proportion to the total counts in each cell were scaled to the intensity value of the “R” or “G” channel in an “RGB” image, respectively. The composite image shows a clear group-dependent distribution of colored cells in space, likely representing two different tumor clones. In a zoomed-in “boundary” region, distinct transcripts from the PC list were detected in different cells (FIG. 13D, ii). Moreover, a supervised dimension reduction was run using the most significantly up-regulated and down-regulated genes from the RNA-seq data of the same PDX model [Campbell, K. R. et al., Genome Biol (2019), Vol. 20(54) 1-12] and the results presented with UMAP, where two tumor cell clusters and one non-tumor cell cluster were identified (FIG. 13D, iii), therefore validating the ExSeq findings.

uniExM for Multimodal Detection Beyond Proteins and Nucleic Acids

Inferred from the reactivity of epoxides, the potential anchoring sites by uniExM in a biological system might consist of a diversity of molecules beyond nucleic acids and proteins. To demonstrate this, studies were carried out to explore the performance of GMA in helping visualization of lipids and carbohydrates (i.e., glycoproteins). Three lipid stains—octadecyl rhodamine B chloride (R18), FM 1-41FX dye (FM) and BODIFY FL C₁₂ (BODIPY) were selected for use. Studies were performed of both pre- and post-expansion staining in which all three lipid-mimic tags gave strong fluorescence signals from membranes and lipid-rich organelles (e.g., mitochondria) (FIG. 14A-B). The strong post-expansion lipid staining signals could be attributed to the anchoring of nucleophilic groups existing on long-chain lipid molecules and preservation of regional hydrophobic domains by uniExM. As a proof of this concept, the selected FM dye contains an anchorable amine group and post-expansion staining with it showed a consistent pattern with pre-expansion staining, indicating successful revelation of certain extent of, if not all, lipid information besides the dye molecules per se anchored by GMA.

Similarly, post-expansion staining was performed using WGA to visualize carbohydrates. The high affinity of WGA to N-acetylglucosamine (GlcNAc) makes it a specific stain for glycoconjugates. Using Alexa647-tagged WGA, GlcNAc-enriched structures including membranes and nucleoporin, can be specifically stained in expanded samples (FIG. 14C). Based on such a broad range of detectable molecules, it was demonstrated that uniExM enabled multi-modal detection within the same sample to examine structural components and functional elements at the same time (FIG. 15A-B). For example, in mouse brain tissues, WGA could be applied to highlight the blood vessels while SMI-312 antibody stained the neurofilaments (FIG. 15C). With in situ contextual information, the location of RNA transcripts can be better interpreted and connected to biological significance.

### Discussion

In order for biomolecules to expand together with the polyacrylate hydrogel in ExM, molecular anchors are needed to connect them. It has been determined that the anchor molecule should therefore be bifunctional, with one end reactive to certain chemical groups on biomolecules and the other end incorporable to the polymer network. In conventional ExM and other tissue processing technologies (e.g., MAP), primary amines are often chosen as the main anchoring point due to the prevalence of lysine in proteins. Hence, in proExM the NHS-containing AcX is employed to covalently anchor proteins, However, such single-target dependent anchoring only preserves protein information while other important molecular information including nucleic acids and carbohydrates are mostly ignored. Although anchoring methods specific to nucleic acids or lipids have been developed, they extensively rely on AcX or customized anchor synthesis where a grand challenge remains for cost control and large room exists for multimodal ExM.

Epoxide is a three-atom, cyclic ether with high reactivity. It is extensively used in polymer production (e.g., resins and adhesives) and its strained “—C—O—C—” triangle structure. makes it an ideal substrate for nucleophilic addition and substitution. Due to its low cost and tunable reactivity, studies were performed to explore the use of acrylate epoxide monomers, a non-limiting example of which is GMA, as a versatile anchor module for uniExM. The epoxide group can react with a number of potent nucleophilic moieties existing on biomolecules and therefore enables covalent linkage of various cellular components to polyacrylate backbone.

Unlike NHS or aldehyde dependent anchoring, epoxides extend intracellular anchorable sites to imidazole, thiol, carboxyl and phenolic hydroxyl groups, going well beyond prior limitations to just primary amines on proteins. Such multivalent anchoring can help achieve more uniform anchoring and better preservation of protein content or epitopes to uniExM. Retention of more epitopes benefits post-expansion staining where fine structures can be resolved with more details and high accuracy, especially under larger expansion factors. GMA is also an efficient anchor molecule to retain nucleic acids on which a few nucleophilic nitrogen atoms reside. The most common reaction site is the guanine N7 position where alkylation could occur through nucleophilic substitution, [Richter, S. et al., Nucleic Acids Res (2003), Vol. 31, 5149-5156; Hansen, M. et at, J Am Chem Soc (1996), Vol. 118, 5553-5561]. Of particular note is that N7-guanine serves as the anchoring site for LabelX as well. Considering the high reactivity of epoxide, it could potentially react with the amine groups on adenine and cytosine if they are properly exposed from DNA (e.g., by denaturation). [Park, Y. G. et at, Nat Biotechnol (2018), Vol. 10(1038)] This would result in a higher labeling efficiency by using epoxides than LabelX for genome probing. The versatile reactivity of epoxides makes it possible to perform multi-modal ExM with a single anchor molecule. As set forth in studies described herein, lipids and carbohydrates signals can be preserved in expanded samples if they were properly processed. The unique anchoring chemistry of epoxides can be used in methods of the invention to explore biological molecules and activities in a composition resembling their original environment.

Recently, another anchor molecule. called MelphaX, was implemented. as an alternative to LabelX in multiplexed HCR-FISH experiments [Wang, Y. et al., Cell (2021), Vol. 184, 6361 -6377]. MelphaX is synthesized by reacting AcX with the nitrogen mustard alkylating agent Melphalan (a chemotherapy medication) and behaves the same as LabelX, in anchoring of nucleic acids. Compared with MelphaX, GMA still holds a multitude of advantages. First and foremost, GMA is the only demonstrated anchor with broad activity that enables controllable retention and multiplexed detection of biomolecules. Second, GMA is more favorable than MelphaX in key physicochemical properties, such as over 60% smaller in size, tunable reactivity (by pH and temperature) and convenient storage (no freezer needed). Third, GMA is a ready-to-use single reagent that does not require customized conjugation or quality evaluation before application. Last but not least, the cost per unit GMA is thousands of times less than that for MelphaX, making it readily to reduce the total cost for as MelphaX-based ExM experiment by more than 50%,

Studies described herein provide evidence of efficacy of methods of the invention in which epoxide is used as an anchor in ExM.

### Equivalents

Although several embodiments of the present invention have been described and illustrated herein, those of ordinary skill in the art will readily envision a variety of other means and/or structures for performing the functions and/or obtaining the results and/or one or more of the advantages described herein, and each of smell variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1. A method for preparing a biospecimen for multimodal detection of a biomolecule, the method comprising:
(a) incubating a biological sample comprising the biomolecule with a multifunctional anchoring agent that covalently bonds the biomolecule, wherein the multifunctional anchoring agent comprises an epoxide compound;
(b) embedding the biological sample with anchored biomolecule in a polymer material;
(c) homogenizing the embedded biological sample; and
(d) physically expanding the homogenized embedded sample.

2. (canceled)

3. The method of claim 1, wherein the biological sample is a fixed biological sample.

4-6. (canceled)

7. The method of claim 1, further comprising performing the method on a plurality of the biomolecules, or on a plurality of different biomolecules.

8. (canceled)

9. The method of claim 1, wherein the epoxide compound comprises an epoxide monomer.

10. The method of claim 1, wherein the epoxide compound comprises glycidyl methacrylate (GMA) and optionally one or more other analogous acrylate epoxides.

11. The method of claim 1, wherein the epoxide compound comprises an epoxide group and an acrylate group.

12. The method of claim 1, wherein the epoxide compound comprises an acrylate epoxide or epoxy acrylate.

13. The method of claim 1, wherein the epoxide compound comprises a precursor molecule that is processed, combined, or conjugated to include epoxide and acrylate groups.

14-30. (canceled)

31. The method of claim 1, further comprising contacting the biomolecule with an antibody, an oligo probe or an affinity label capable of selectively binding the biomolecule, at one or both of before or after the physical expansion step.

32. The method of claim 1, further comprising attaching the biomolecule to a solid support prior to the embedding step.

33-40. (canceled)

41. The method of claim 1, further comprising detecting one or more of a spatial position, a structure, a component of, and an identity of the expanded biomolecule(s).

42-44. (canceled)

45. The method of claim 41, wherein a means for the detecting comprises a method capable of capturing spatial data.

46. The method of claim 41, wherein a means for the detecting comprises transferring the homogenized biomolecule from the polymer to a spatially indexed array, wherein the spatially indexed array optionally comprises a microarray or a bead array.

47. The method of claim 41, wherein a means for the detecting comprises sectioning the expanded biomolecule, identifying the relative positions of the sections, recovering homogenized biomolecule material from the sections, detecting the homogenized biomolecule material, associating the detected homogenized biomolecule material with the identified relative positions of the homogenized biomolecule material versus the non-homogenized biomolecule, and determining spatial positions of the associated detected homogenized biomolecule material.

48-53. (canceled)

54. The method of claim 1, further comprising detectably labeling the biomolecule.

55-58. (canceled)

59. The method of claim 41, wherein one or more of a detected spatial position, presence, absence, components of, and level of the biomolecule is associated with a disease or condition.

60. The method of claim 41, further comprising classifying one or more of the detected spatial position, structure, and component of the expanded biomolecule(s) into one or more contiguous biomolecule molecule.

61. The method of claim 60, wherein a means of the classifying comprises identifying the spatial positions of the detected homogenized biomolecule material in one or more dimensions and determining a relative ordering of the detected homogenized biomolecule material within a single contiguous biomolecule, wherein the relative ordering aids in classifying the detected homogenous biomolecule material into one or more contiguous biomolecules and identifying a structure of the ordered contiguous biomolecule.

62. The method of claim 61, further comprising identifying one or more of a spatial position and a structural variation in the one or more classified contiguous biomolecules compared to a control structure.

63. (canceled)

64. The method of claim 1, wherein the biomolecule is obtained from a cell.

65-68. (canceled)


SEQUENCE LISTING (1 sequence)

SEQ ID NO: 1 - DNA, 22 bp, synthetic construct
  source 1..22: mol_type = other DNA
tctcgggaacgctgaagacggc